(19) United States
(12) Patent Application Publication
(10) Pub. No.: US 2005/0192441 A1
(43) Pub. Date: Sep. 1, 2005

(54) Methods for the preparation of stereoisomerically enriched amines

(75) Inventors: Shanghui Hu, San Diego, CA (US); Carlos Martinez, Oceanside, CA (US); Junhua Tao, San Diego, CA (US); Daniel Rida Yazbeck, San Diego, CA (US)

Correspondence Address: AGOURON PHARMACEUTICALS, INC., 10777 SCIENCE CENTER DRIVE, SAN DIEGO, CA, 92121, US

(73) Assignee: Agouron Pharmaceuticals, Inc.

(21) Appl. No.: 11/003,830

(22) Filed: Dec. 3, 2004

Related U.S. Application Data
(60) Provisional application No. 60/527,143, filed on Dec. 4, 2003.

(51) Int. Cl. C07D 417/02; C07D 277/18

(52) U.S. Cl. 548/200; 548/530

(57) ABSTRACT

The present invention relates to methods of preparing a stereoisomerically enriched compound of formula (I), 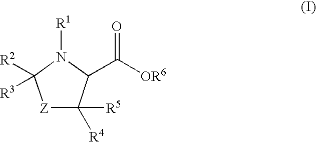
wherein R⁶ is hydrogen, comprising treating a compound of formula (I), wherein R⁶ is chosen from C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁸), with a biocatalyst in an aqueous solution, an organic solvent, or a mixture of organic and aqueous solvents wherein at least one stereoisomer is selectively hydrolyzed.

This application claims priority under 35 U.S.C. § 119(e) to U.S. Patent Application No. 60/527,143, filed Dec. 4, 2003, which is hereby incorporated by reference.

### BACKGROUND

The present invention relates to methods for the preparation of stereoisomerically enriched amines. The stereoisomerically enriched amines disclosed herein are useful in the preparation of compounds that inhibit the Human Immunodeficiency virus (HIV) protease enzyme.

Acquired Immune Deficiency Syndrome (AIDS) causes a gradual breakdown of the body's immune system as well as progressive deterioration of the central and peripheral nervous systems. Since its initial recognition in the early 1980's, AIDS has spread rapidly and has now reached epidemic proportions within a relatively limited segment of the population. Intensive research has led to the discovery of the responsible agent, human T-lymphotropic retrovirus III (HTLV-III), now more commonly referred to as HIV.

HIV is a member of the class of viruses known as retroviruses and is the etiologic agent of AIDS. The retroviral genome is composed of RNA, which is converted to DNA by reverse transcription. This retroviral DNA is then stably integrated into a host cell's chromosome and, employing the replicative processes of the host cells, produces new retroviral particles and advances the infection to other cells. HIV appears to have a particular affinity for the human T-4 lymphocyte cell, which plays a vital role in the body'simmune system. HIV infection of these white blood cells depletes this white cell population. Eventually, the immune system is rendered inoperative and ineffective against various opportunistic diseases such as, among others, pneumocystic carini pneumonia, Kaposi's sarcoma, and cancer of the lymph system.

Although the exact mechanism of the formation and working of the HIV virus is not understood, identification of the virus has led to some progress in controlling the disease. For example, the drug azidothymidine (AZT) has been found effective for inhibiting the reverse transcription of the retroviral genome of the HIV virus, thus giving a measure of control, though not a cure, for patients afflicted with AIDS. The search continues for drugs that can cure or at least provide an improved measure of control of the deadly HIV virus and thus the treatment of AIDS and related diseases.

Retroviral replication routinely features post-translational processing of polyproteins. This processing is accomplished by virally encoded HIV protease enzyme. This yields mature polypeptides that will subsequently aid in the formation and function of infectious virus. If this molecular processing is stifled, then the normal production of HIV is terminated. Therefore, inhibitors of HIV protease may function as anti-HIV viral agents.

HIV protease is one of the translated products from the HIV structural protein pol 25 gene. This retroviral protease specifically cleaves other structural polypeptides at discrete sites to release these newly activated structural proteins and enzymes, thereby rendering the virion replication-competent. As such, inhibition of the HIV protease by potent compounds may prevent proviral integration of infected T-lymphocytes during the early phase of the HIV-1 life cycle, as well as inhibit viral proteolytic processing during its late stage. Additionally, the protease inhibitors may have the advantages of being more readily available, longer lived in virus, and less toxic than currently available drugs, possibly due to their specificity for the retroviral protease.

Methods for preparing compounds useful as HIV protease inhibitors have been described in, e.g., U.S. Pat. No. 5,962,640; U.S. Pat. No. 5,932,550; U.S. Pat. No. 6,222,043; U.S. Pat. No. 5,644,028; WO 02/100844, Australian Patent No. 705193; Canadian Patent Application No. 2,179,935; European Patent Application No. 0 751 145; Japanese Patent Application No. 100867489; Y. Hayahsi, et al., J. Org. Chem., 66, 5537-5544 (2001); K. Yoshimura, et al., Proc. Natl. Acad. Sci. USA, 96, 8675-8680 (1999); and, T. Mimoto, et al., J. Med. Chem., 42, 1789-1802 (1999). Many HIV protease inhibitors are complex molecules that contain one or more asymmetric carbon atoms. Preparing such compounds in an efficient and stereochemically selective manner is a challenge facing synthetic chemists. Although some stereoselective synthetic methods have been developed to address these needs, they are generally not applicable to a wide range of molecules. As such, the need still exists for the development of stereoselective methods that can be used to efficiently prepare such complex molecules.

### SUMMARY

The present invention relates to methods of preparing a stereoisomerically enriched compound of formula (I), 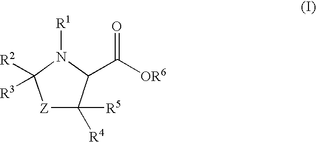
wherein:
Z is O, S, C═O, C═CH₂, or —(CR⁷R⁸)—;
R¹ is hydrogen, —(CR⁷R⁸)ₜ(C₆-C₁₄ aryl), —CH₂CH═CH₂, —C(O)R⁷, —C(O)OR⁷, —C(O)C(O)OR⁷, or —Si(R⁷)₃, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen, C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁷, and —N(R⁷R⁸);
R⁴ and R⁵ are independently chosen from hydrogen, halo, C₁-C₁₀ alkyl, C₂-C₁₀ alkenyl, C₂-C₁₀ alkynyl, —(CR⁷R₈)ₜ(C₆-C₁₄ aryl), and —(CR⁷R)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁷, and —N(R⁷R⁸);
R⁶ is hydrogen;
each R⁷ and R⁸ is independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 0 to 5;
   said method comprising:
treating a compound of formula (I), wherein R¹, R², R³, R⁴ and R⁵ are as defined above and R⁶ is chosen from C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁸), with a biocatalyst in an aqueous solution, an organic solvent, or a mixture of organic and aqueous solvents wherein at least one stereoisomer is selectively hydrolyzed.

Another aspect of the present invention provides any of the methods described herein of preparing a stereoisomerically enriched compound of formula (I), wherein:
Z is O, S, C═O, C═CH₂, or —(CR⁷R⁸)—;
R¹ is —(CR⁷R⁸)ₜ(C₆-C₁₄ aryl), —CH₂CH═CH₂, —C(O)R⁷, —C(O)OR⁷, —C(O)C(O)OR⁷, or —Si(R⁷)₃, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen and C₁-C₁₀ alkyl;
R⁴ and R⁵ are independently chosen from hydrogen, halo, and C₁-C₁₀ alkyl;
R⁶ is hydrogen;
R⁷ and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 0 to 5.

In still another aspect of the present invention are provided any of the methods described herein of preparing a stereoisomerically enriched compound of formula (I), wherein:
Z is O, S, C═O, C═CH₂, or —(CR⁷R⁸)—;
R¹ is —(CH₂)ₜ(C₆-C₁₄ aryl), —CH₂CH═CH₂, —C(O)OR⁷, or —C(O)C(O)OR⁷, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen and C₁-C₁₀ alkyl;
R⁴ and R⁵ are independently chosen from hydrogen, halo, and C₁-C₁₀ alkyl;
R⁶ is hydrogen;
R⁷ and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 1 to 3.

In another aspect of the present invention are provided any of the methods described herein of preparing a stereoisomerically enriched compound of formula (I), wherein:
Z is O, S, C═O, C═CH₂, or —(CR⁷R⁸)—,
R¹ is —(CH₂)(C₆-C₁₄ aryl), —CH₂CH═CH₂, —C(O)OR⁷, or —C(O)C(O)OR⁷, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen, methyl, ethyl, butyl, and pentyl;
R⁴ and R⁵ are independently chosen from hydrogen, halo, methyl, ethyl, butyl, and pentyl;
R⁶ is hydrogen;
R⁷ and R⁸ are independently chosen from hydrogen, halo, C₁-C₁₀ alkyl, C₁-C₁₀ alkoxy, C₂-C₁₀ alkenyl, and C₆-C₁₄ aryl, wherein said C₆-C₁₄ aryl is optionally substituted with at least one substituent chosen from halo, C₁-C₁₀ alkyl, —OR⁹, and —N(R⁹R⁹); and
each R⁹ is independently chosen from hydrogen and C₁-C₁₀ alkyl.

In still a further aspect of the present invention are provided any of the methods described herein of preparing a stereoisomerically enriched compound of formula (I), wherein:
Z is O, S, C═O, C═CH₂, or —(CR⁷R⁸)—;
R¹ is —CH₂Ph, —C(O)OR⁷, or —C(O)C(O)OR⁷;
R² and R³ are hydrogen;
R⁴ and R⁵ are independently chosen from hydrogen and methyl;
R⁶ is hydrogen; and
R⁷ and R⁸ are independently chosen from hydrogen and C₁-C₁₀ alkyl.

The present invention also provides any of the methods described herein of preparing a stereoisomerically enriched compound of formula (I), wherein:
Z is O, S, C═O, C═CH₂, or —(CR⁷R⁸)—;
R¹ is —CH₂Ph, —C(O)OCH₃, —C(O)OC(CH₃)₃, or —C(O)C(O)OCH₃;
R² and R³ are hydrogen;
R⁴ and R⁵ are independently chosen from hydrogen and methyl;
R⁶ is hydrogen; and
R⁷ and R⁸ are independently chosen from hydrogen, fluorine, methyl, and —OCH₃.

In another aspect of the present invention are provided any of the methods described herein of preparing a stereoisomerically enriched compound of formula (I), wherein:
Z is S;
R¹ is —CH₂Ph, —C(O)OCH₃, —C(O)OC(CH₃)₃, or —C(O)C(O)OCH₃;
R² and R³ are hydrogen;
R⁴ and R⁵ are methyl; and
R⁶ is hydrogen.

In still a further aspect of the present invention are provided any of the methods described herein of preparing a stereoisomerically enriched compound of formula (I), wherein:
Z is C═O, C═CH₂, or —(CR⁷R⁸)—;

R¹ is —(CR⁷R⁸)ₜ(C₆-C₁₄ aryl), —CH₂CH═CH₂, —C(O)R⁷, —C(O)OR⁷, —C(O)C(O)OR⁷, or —Si(R⁷)₃, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen and C₁-C₁₀ alkyl;
R⁴ and R⁵ are independently chosen from hydrogen, halo, and C₁-C₁₀ alkyl;
R⁶ is hydrogen;
R⁷ and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 0 to 5.

Another aspect of the present invention provides any of the methods described herein of preparing a stereoisomerically enriched compound of formula (I), wherein:
Z is C═O, C═CH₂, or —(CR⁷R⁸)—;
R¹ is —CH₂Ph, —CH₂CH═CH₂, —C(O)R⁷, —C(O)OR⁷, or —C(O)C(O)OR⁷;
R² and R³ are independently chosen from hydrogen and C₁-C₁₀ alkyl;
R⁴ and R⁵are independently chosen from hydrogen, halo, and C₁-C₁₀ alkyl;
R⁶is hydrogen;
R⁷ and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 1 to 3.

In still a further aspect of the present invention are provided any of the methods described herein s of preparing a stereoisomerically enriched compound of formula (I), wherein:
Z is —(CR⁷R⁸)—;
R¹ is —CH₂Ph, —CH₂CH═CH₂, —C(O)OCH₃, —C(O)OC(CH₃)₃, or —C(O)C(O)OCH₃;
R² and R³ are hydrogen;
R⁴ and R⁵ are independently chosen from hydrogen and methyl, ethyl, butyl and pentyl;
R⁶ is hydrogen; and
R⁷ and R⁸ are independently chosen from hydrogen, fluorine, chlorine, C₁-C₁₀ alkyl, and C₁-C₁₀ alkoxy.

The present invention further relates to methods of preparing a stereoisomerically enriched compound of formula (IA), 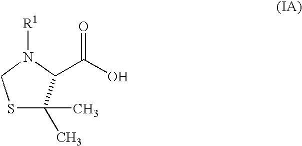
wherein:
R¹ is —CH₂Ph, —C(O)OR⁷, or —C(O)C(O)OR⁷; and
R⁷ is C₁-C₁₀ alkyl;
   said method comprising:
treating a compound of formula (IC), 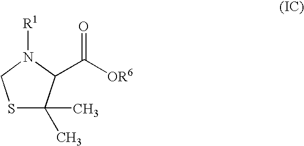
wherein R¹ is as defined above and R⁶ is chosen from C₁-C₁₀ alkyl, C₂-C₁₀ alkenyl, C₂-C₁₀ alkynyl, —CH₂(C₆-C₁₄ aryl), and —CH₂(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁷), with a biocatalyst in an aqueous solution, an organic solvent, or a mixture of organic and aqueous solvents wherein at least one stereoisomer is selectively hydrolyzed.

The present invention further relates to any of the methods described herein of preparing a stereoisomerically enriched compound of formula (IA), wherein R¹ is —C(O)OC(CH₃)₃, comprising treating a compound of formula (IC), wherein R¹ is —C(O)OC(CH₃)₃ and R⁶ is —CH₃, with a biocatalyst in an aqueous solution, an organic solvent, or a mixture of organic and aqueous solvents wherein at least one stereoisomer is selectively hydrolyzed.

In still a further aspect of the present invention are provided any of the methods described herein of preparing a stereoisomerically enriched compound of formula (IA), wherein R¹ is —CH₂Ph, comprising treating a compound of formula (IC), wherein R¹ is —CH₂Ph and R⁶ is —CH₃, with a biocatalyst in an aqueous solution, an organic solvent, or a mixture of organic and aqueous solvents wherein at least one stereoisomer is selectively hydrolyzed.

In another aspect of the present invention are provided methods of preparing a stereoisomerically enriched compound of formula (IB), 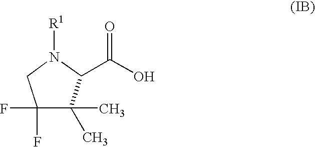
wherein:
R¹ is —CH₂Ph, —C(O)OR⁷, or —C(O)C(O)OR⁷; and
R⁷ is C₁-C₁₀ alkyl;
   said method comprising:
treating a compound of formula (ID), 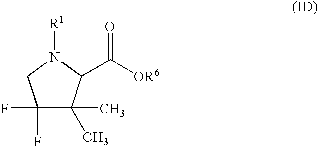
   wherein R¹ is as defined above and R⁶ is chosen from C₁-C₁₀ alkyl, C₂-C₁₀ alkenyl, C₂-C₁₀ alkynyl, —CH₂(C₆-C₁₄ aryl), and —CH₂(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁷), with a biocatalyst in an aqueous solution, an organic solvent, or a mixture of organic and aqueous solvents wherein at least one stereoisomer is selectively hydrolyzed.

The present invention further relates to any of the methods described herein of preparing a stereoisomerically enriched compound of formula (IB), wherein R¹ is —C(O)OC(CH₃)₃, comprising treating a compound of formula (ID), wherein R¹ is —C(O)OC(CH₃)₃ and R⁶ is —CH₃, with a biocatalyst in an aqueous solution, an organic solvent, or a mixture of organic and aqueous solvents wherein at least one stereoisomer is selectively hydrolyzed.

In still a further aspect of the present invention are provided any of the methods described herein of preparing stereoisomerically enriched compound of formula (IB), wherein R¹ is —CH₂Ph, comprising treating a compound of formula (ID), wherein R¹ is —CH₂Ph and R⁶ is —CH₃, with a biocatalyst in an aqueous solution, an organic solvent, or a mixture of organic and aqueous solvents wherein at least one stereoisomer is selectively hydrolyzed.

Also provided in the present invention are any of the methods described herein of preparing stereoisomerically enriched compounds of formulas (I), (IA), and (IB), wherein said biocatalyst is chosen from an alkaline protease, an esterase, a lipase, a hydrolase, and any combination thereof. In another aspect of the present invention are provided methods wherein said biocatalyst is chosen from Klebsiella oxytoca, Aspergillus melleus, Bacilius subtilis, Bacillus licheniformis, Bacillus lentus, and Pig Liver esterase.

The present invention also relates to a method for the resolution of a compound of formula (I), 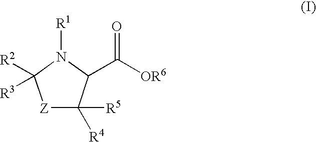
wherein:
Z is O, S, C═O, C═CH₂, or —(CR⁷R⁸)—;
R¹ is hydrogen, —(CR⁷R⁸)ₜ(C₆-C₁₄ aryl), —CH₂CH═CH₂, —C(O)R⁷, —C(O)OR⁷, —C(O)C(O)OR⁷, or —Si(R⁷)₃, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen, C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁷, and —N(R⁷R⁸);
R⁴ and R⁵ are independently chosen from hydrogen, halo, C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁷, and —N(R⁷R⁸);
R⁶ is chosen from C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁸);
R⁷ and R⁸ are independently chosen from hydrogen, halo, C₁-C₁₀ alkyl, C₁-C₁₀ alkoxy, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 0 to 5;
   said method comprising:
treating the compound of formula (I) with a biocatalyst in an aqueous solvent, an organic solvent, or a mixture of aqueous and organic solvents, to afford a stereoisomerically enriched compound of formula (I) wherein R⁶ is hydrogen.

Another aspect of the present invention provides any of the methods described herein for the resolution of a compound of formula (I), wherein:
Z is O, S, C═O, C═CH₂, or —(CR⁷R⁸)—;

R¹ is —(CR⁷R⁸)ₜ(C₆-C₁₄ aryl), —CH₂CH═CH₂, —C(O)R⁷, —C(O)OR⁷, —C(O)C(O)OR⁷, or —Si(R⁷)₃, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen and C₁-C₁₀ alkyl
R⁴ and R⁵ are independently chosen from hydrogen, halo, and C₁-C₁₀ alkyl;
R⁶ is hydrogen;
R⁷ and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 0 to 5.

In still another aspect of the present invention are provided any of the methods described herein for the resolution of a compound of formula (I), wherein:
Z is O, S, C═O, C═CH₂, or —(CR⁷R⁸)—;
R¹ is —(CH₂)ₜ(C₆-C₁₄ aryl), —CH₂CH═CH₂, —C(O)OR⁷, or —C(O)C(O)OR⁷, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen and C₁-C₁₀ alkyl;
R⁴ and R⁵ are independently chosen from hydrogen, halo, and C₁-C₁₀ alkyl;
R⁶ is hydrogen;
R⁷and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 1 to 3.

In another aspect of the present invention are provided any of the methods described herein for the resolution of a compound of formula (I), wherein:
Z is O, S, C═O, C═CH₂, or —(CR⁷R⁸)—;
R¹ is —(CH₂)(C₆-C₁₄ aryl), —CH₂CH═CH₂, —C(O)OR⁷, or —C(O)C(O)OR⁷, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen, methyl, ethyl, butyl, and pentyl;
R⁴ and R⁵ are independently chosen from hydrogen, halo, methyl, ethyl, butyl, and pentyl;
R⁶is hydrogen;
R⁷ and R⁸ are independently chosen from hydrogen, halo, C₁-C₁₀ alkyl, C₁-C₁₀ alkoxy, C₂-C₁₀ alkenyl, and C₆-C₁₄ aryl, wherein said C₆-C₁₄ aryl is optionally substituted with at least one substituent chosen from halo, C₁-C₁₀ alkyl, —OR⁹, and —N(R⁹R⁹); and
each R⁹ is independently chosen from hydrogen and C₁-C₁₀ alkyl.

In still a further aspect of the present invention are provided any of the methods described herein for the resolution of a compound of formula (I), wherein:
Z is O, S, C═O, C═CH₂, or —(CR⁷R⁸)—;
R¹ is —CH₂Ph, —C(O)OR⁷, or —C(O)C(O)OR⁷;
R² and R³ are hydrogen;
R⁴ and R⁵ are independently chosen from hydrogen and methyl;
R⁶ is hydrogen; and
R⁷ and R⁸ are independently chosen from hydrogen, halo, C₁-C₁₀ alkyl, and C₁-C₁₀ alkoxy.

The present invention also provides any of the methods described herein for the resolution of a compound of formula (I), wherein:
Z is O, S, C═O, C═CH₂, or —(CR⁷R⁸)—;
R¹ is —CH₂Ph, —C(O)OCH₃, —C(O)OC(CH₃)₃, or —C(O)C(O)OCH₃;
R² and R³ are hydrogen;
R⁴ and R⁵ are independently chosen from hydrogen and methyl;
R⁶ is hydrogen; and
R⁷ and R⁸ are independently chosen from hydrogen, fluorine, methyl, and —OCH₃.

In another aspect of the present invention are provided any of the methods described herein for the resolution of a compound of formula (I), wherein:
Z is S;
R¹ is —CH₂Ph, —C(O)OCH₃, —C(O)OC(CH₃)₃, or —C(O)C(O)OCH₃;
R² and R³ are hydrogen;
R⁴ and R⁵ are methyl; and
R⁶ is hydrogen.

In still a further aspect of the present invention are provided any of the methods described herein for the resolution of a compound of formula (I), wherein:
Z is C═O, C═CH₂, or —(CR⁷R⁸)—;

R¹ is —(CR⁷R⁸)ₜ(C₆-C₁₄ aryl), —CH₂CH═CH₂, —C(O)R⁷, —C(O)OR⁷, —C(O)C(O)OR⁷, or —Si(R⁷)₃, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen and C₁-C₁₀ alkyl;
R⁴ and R⁵are independently chosen from hydrogen, halo, and C₁-C₁₀ alkyl;
R⁶ is hydrogen;
R⁷ and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 0 to 5.

Another aspect of the present invention provides any of the methods described herein for the resolution of a compound of formula (I), wherein:
Z is C═O, C═CH₂, or —(CR⁷R⁸)—;
R¹ is —CH₂Ph, —CH₂CH═CH₂, —C(O)R⁷, —C(O)OR⁷, or —C(O)C(O)OR⁷;
R² and R³ are independently chosen from hydrogen and C₁-C₁₀ alkyl;
R⁴ and R⁵ are independently chosen from hydrogen, halo, and C₁-C₁₀ alkyl;
R⁶ is hydrogen;
R⁷ and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 1 to 3.

In still a further aspect of the present invention are provided any of the methods described herein for the resolution of a compound of formula (I), wherein:
Z is —(CR⁷R⁸)—;
R¹ is —CH₂Ph, —CH₂CH═CH₂, —C(O)OCH₃, —C(O)OC(CH₃)₃, or —C(O)C(O)OCH₃;
R² and R³ are hydrogen;
R⁴ and R⁵ are independently chosen from hydrogen and methyl, ethyl, butyl and pentyl;
R⁶ is hydrogen; and
R⁷ and R⁸ are independently chosen from hydrogen, fluorine, chlorine, C₁-C₁₀ alkyl, and C₁-C₁₀ alkoxy.

Another aspect of the present invention provides a method of preparing a stereoisomerically enriched compound of formula (II), 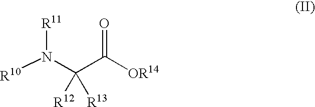
wherein:

R¹⁰ is hydrogen, —(CR¹⁵R¹⁶)ₜ(C₆-C₁₄ aryl), —CH₂CH═CH₂, —C(O)R¹⁵, —C(O)OR¹⁵, —C(O)C(O)OR¹⁵, or —Si(R¹⁵)₃, wherein said C₆-C₁₄ aryl is optionally substituted with at least one substituent chosen from halo, C₁-C₁₀ alkyl, —OR¹⁵, and —N(R¹⁵R¹⁶);

R¹¹ is hydrogen, C₁-C₁₀ alkyl, C₂-C₁₀ alkenyl, C₂-C₁₀ alkynyl, —(CR¹⁵R¹⁶)ₜ(C₆-C₁₄ aryl), or —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶);
R¹² and R¹³ are independently chosen from hydrogen, C₁-C₁₀ alkyl, C₂-C₁₀ alkenyl, C₂-C₁₀ alkynyl, —(CR¹⁵R¹⁶)ₜ(C₆-C₁₄ aryl), and —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶), provided that R¹² and R¹³ cannot both be hydrogen;
R¹⁴ is hydrogen;
R¹⁵ and R¹⁶ are independently chosen from hydrogen, halo, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5;
   said method comprising:
treating a compound of formula (II), wherein R¹⁰, R¹¹, R¹², and R¹³ are as defined above, and R¹⁴ is chosen from C₁-C₁₀ alkyl, C₂-C₁₀ alkenyl, C₂-C₁₀ alkynyl, —(CR¹⁵R¹⁶)ₜ(C₆-C₁₄ aryl), and —(CR¹⁵R¹⁶)ₜ(4-10 membered heterocyclic), and wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶), with a biocatalyst in an aqueous solution, an organic solvent, or a mixture of organic and aqueous solvents wherein at least one stereoisomer is selectively hydrolyzed.

In still a further aspect of the present invention are provided any of the methods described herein of preparing a stereoisomerically enriched compound of formula (II), wherein:
R¹⁰ is —(CR¹⁵R¹⁶)ₜ(C₆-C₁₄ aryl), —C(O)OR¹⁵, or —C(O)C(O)OR¹⁵, wherein said C₆-C₁₄ aryl is optionally substituted with at least one substituent chosen from halo, C₁-C₁₀ alkyl, —OR¹⁵, and —N(R¹⁵R¹⁶);

R¹¹ is hydrogen, C₁-C₁₀ alkyl, C₂-C₁₀ alkenyl, C₂-C₁₀ alkynyl, —(CR¹⁵R¹⁶)ₜ(C₆-C₁₄ aryl ), or —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶);
R¹² and R¹³ are independently chosen from hydrogen, C₁-C₁₀ alkyl, C₂-C₁₀ alkenyl, C₂-C₁₀ alkynyl, —(CR¹⁵R¹⁶ )ₜ(C₆-C₁₄ aryl), and —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶), provided that R¹² and R¹³ cannot both be hydrogen;
R¹⁴ is hydrogen;
R¹⁵ and R¹⁶ are independently chosen from hydrogen, halo, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5.

In yet another aspect of the present invention are provided any of the methods described herein of preparing a stereoisomerically enriched compound of formula (II), wherein:
R¹⁰ is —C(O)OR¹⁵ or —C(O)C(O)OR¹⁵;
R¹¹ is hydrogen or C₁-C₁₀ alkyl;
R¹² and R¹³ are independently chosen from hydrogen, C₁-C₁₀ alkyl, C₂-C₁₀ alkenyl, and C₂-C₁₀ alkynyl, provided that R¹² and R¹³ cannot both be hydrogen;
R¹⁴ is hydrogen; and
R¹⁵ is C₁-C₁₀ alkyl.

In still a further aspect of the present invention are provided any of the methods described herein of preparing a stereoisomerically enriched compound of formula (II), wherein:
R¹⁰ is —C(O)OR¹⁵ or —C(O)C(O)OR¹⁵;
R¹¹ is hydrogen;
R¹² is hydrogen;
R¹³ is C₂-C₁₀ alkenyl;
R¹⁴ is hydrogen; and
R¹⁵ is C₁-C₁₀ alkyl.

Another aspect of the present invention provides any of the methods described herein of preparing a stereoisomerically enriched compound of formula (II), wherein:
R¹⁰ is —C(O)OR¹⁵ or —C(O)C(O)OR¹⁵;
R¹¹ is hydrogen;
R¹² is hydrogen;
R¹³ is C₂-C₅ alkenyl;
R¹⁴ is hydrogen; and
R¹⁵ is —C(CH₃)₃.

The present invention also relates to methods of preparing a stereoisomerically enriched compound of formula (IIA), 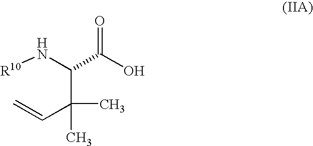
wherein:
R¹⁰ is chosen from hydrogen, —(CR¹⁵R¹⁶)ₜ(C₆-C₁₄ aryl), —CH₂CH═CH₂, —C(O)R¹⁵, —C(O)OR¹⁵, and —C(O)C(O)OR¹⁵; and
each R¹⁵ and R¹⁶ are independently chosen from hydrogen, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5;
   said method comprising:
treating a compound of formula (IIB), 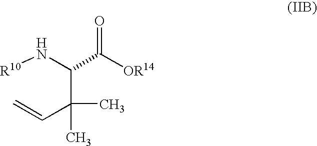
   wherein R¹⁰ is as defined above, and R¹⁴ is C₁-C₁₀ alkyl, with a biocatalyst in an aqueous solution, an organic solvent, or a mixture of organic and aqueous solvents wherein at least one stereoisomer is selectively hydrolyzed.

Also provided in the present invention are any of the methods described herein of preparing a stereoisomerically enriched compound of formula (IIA), wherein R¹⁰ is —C(O)OC(CH₃)₃, said method comprising, treating a compound of formula (IIB), wherein R¹⁰ is as defined above and R¹⁴ is methyl, with a biocatalyst in an aqueous solution, an organic solvent, or a mixture of organic and aqueous solvents wherein at least one stereoisomer is selectively hydrolyzed.

Also provided in the present invention are any of the methods described herein of preparing stereoisomerically enriched compounds of formulas (II) and/or (IIA), wherein said biocatalyst is chosen from an alkaline protease, an esterase, a lipase, a hydrolase, and any combination thereof. In another aspect of the present invention are provided methods wherein said biocatalyst is chosen from Klebsiella oxytoca, Aspergillus melleus, Bacilius subtilis, and Pig Liver esterase.

In still another aspect of the present invention are provided methods for the resolution of a compound of formula (II), 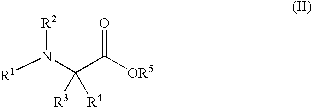
wherein:
R¹ is hydrogen, —(CR⁷R⁸)ₜ(C₆-C₁₄ aryl), —CH₂CH═CH₂, —C(O)R⁷, —C(O)OR⁷, —C(O)C(O)OR⁷, or —Si(R₇)₃, wherein said C₆-C₁₄ aryl is optionally substituted with at least one substituent chosen from halo, C₁-C₁₀ alkyl, —OR⁷, and —N(R⁷R⁸);
R² is hydrogen, C₁-C₁₀ alkyl, C₂-C₁₀ alkenyl, C₂-C₁₀ alkynyl, —(CR⁷R⁸)ₜ(C₆-C₁₄ aryl ), or —(CR⁷R⁸)ₜ(4-10 membered heterocyclic), wherein said C₆-C₁₄ aryl and 4-10 membered heterocyclic are optionally substituted with at least one substituent chosen from halo, C₁-C₁₀ alky, —OR⁷, and —N(R⁷R⁸);
R³ and R⁴ are independently chosen from hydrogen, C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁷, and —N(R⁷R⁸), provided that R³ and R⁴ cannot both be hydrogen;
R⁵ is chosen from C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁸);
R⁷ and R⁸ are independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 0 to 5;
   said method comprising:
treating the compound of formula (II) with a biocatalyst in an aqueous solvent, an organic solvent, or a mixture of aqueous and organic solvents, to afford a stereoisomerically enriched compound of formula (II) wherein R⁵ is hydrogen.

In still a further aspect of the present invention are provided methods of preparing a stereoisomerically enriched compound of formula (II), 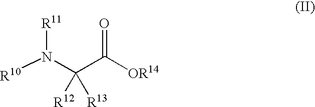
wherein:
R¹⁰ is hydrogen, —(CR¹⁵R¹⁶)ₜ(C₆-C₁₄ aryl), —CH₂CH═CH₂, —C(O)R¹⁵, —C(O)OR¹⁵, —C(O)C(O)OR¹⁵, or —Si(R¹⁵)₃, wherein said C₆-C₁₄ aryl is optionally substituted with at least one substituent chosen from halo, C₁-C₁₀ alkyl, —OR¹⁵, and —N(R¹⁵R¹⁶);
R¹¹ is hydrogen, C₁-C₁₀ alkyl, C₂-C₁₀ alkenyl, C₂-C₁₀ alkynyl, —(CR¹⁵R¹⁶)ₜ(C₆-C₁₄ aryl), or —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶);
R¹² and R¹³ are independently chosen from hydrogen, C₁-C₁₀ alkyl, C₂-C₁₀ alkenyl, C₂-C₁₀ alkynyl, —(CR¹⁵R¹⁶)ₜ(C₆-C₁₄ aryl), and —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶), provided that R¹² and R¹³ cannot both be hydrogen;
R¹⁴ is hydrogen;
R¹⁵ and R¹⁶ are independently chosen from hydrogen, halo, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5;
   said method comprising:
(i) treating a compound of formula (II), wherein R¹⁰, R¹¹, R¹², R¹³, and R¹⁴ are as defined above, with a chiral, non-racemic base to afford a mixture of diastereomeric salts;
(ii) separating said diastereomeric salts from each other; and
(iii) converting said diastereomeric salt to a stereoisomerically enriched compound of formula (II).

In yet another aspect of the present invention are provided methods for the resolution of a compound of formula (II), 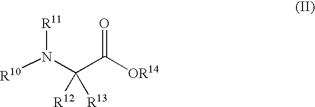
wherein:
R¹⁰ is hydrogen, —(CR¹⁵R¹⁶)ₜ(C₆-C₁₄ aryl), —CH₂CH═CH₂, —C(O)R¹⁵, —C(O)OR¹⁵, —C(O)C(O)OR¹⁵, or —Si(R¹⁵)₃, wherein said C₆-C₁₄ aryl is optionally substituted with at least one substituent chosen from halo, C₁-C₁₀ alkyl, —OR¹⁵, and —N(R¹⁵R¹⁶);
R¹¹ is hydrogen, C₁-C₁₀ alkyl, C₂-C₁₀ alkenyl, C₂-C₁₀ alkynyl, —(CR¹⁵R¹⁵)ₜ(C₆-C₁₄ aryl), or —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶);
R¹² and R¹³ are independently chosen from hydrogen, C₁-C₁₀ alkyl, C₂-C₁₀ alkenyl, C₂-C₁₀ alkynyl, —(CR¹⁵R¹⁶)ₜ(C₆-C₁₄ aryl), and —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶), provided that R¹² and R¹³ cannot both be hydrogen;
R¹⁴ is hydrogen;
R¹⁵ and R¹⁶ are independently chosen from hydrogen, halo, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5;
   said method comprising:
(i) treating a compound of formula (II), wherein R¹⁰, R¹¹, R¹², R¹³, and R¹⁴ are as defined above, with a chiral, non-racemic base to afford a mixture of diastereomeric salts;
(ii) separating said diastereomeric salts from each other; and
(iii) converting said diastereomeric salt to a stereoisomerically enriched compound of formula (II).

The present invention also relates to methods of resolving a compound of formula (IIA), 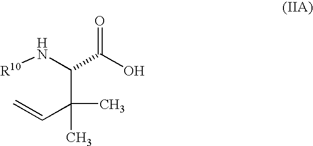
wherein:
R¹⁰ is chosen from hydrogen, —(CR¹⁵R¹⁶)ₜ(C₆-C₁₄ aryl), —CH₂CH═CH₂, —C(O)R¹⁵, —C(O)OR¹⁵, and —C(O)C(O)OR¹⁵; and
each R¹⁵ and R¹⁶ are independently chosen from hydrogen, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5;
   said method comprising:
(i) treating a compound of formula (IIA), wherein R¹⁰ is as defined above, with a chiral, non-racemic base to afford a mixture of diastereomeric salts;
(ii) separating said diastereomeric salts from each other; and
(iii) converting said diastereomeric salt to a stereoisomerically enriched compound of formula (IIA).

Also provided in the present invention are any of the methods described herein of resolving a compound of formula (IIA), wherein R¹⁰ is —C(O)OC(CH₃)₃.

Also included in the present invention are any of the methods described herein of resolving a compound of formula (IIA), wherein said chiral, non-racemic base is either (R)-(−)-2-phenylglycinol or (S)-(+)-2-phenylglycinol. Other resolving agents useful in the present invention include other chiral, non-racemic amines including, but not limited to, either enantiomer of 2-amino-1-phenyl-1,3-propanediol and either enantiomer of 1-phenyl-1-aminoethane.

As used herein, the terms “comprising” and “including” are used in their open, non-limiting sense.

As used herein, the term “HIV” means Human Immunodeficiency Virus. The term “HIV protease,” as used herein, means the Human Immunodeficiency Virus protease enzyme.

The term “C₁-C₁₀ alkyl”, as used herein, unless otherwise indicated, includes saturated monovalent hydrocarbon radicals having straight, branched, or cyclic moieties (including fused and bridged bicyclic and spirocyclic moieties), or a combination or the foregoing moieties, and containing from 1-10 carbon atoms. For an alkyl group to have cyclic moieties, the group must have at least three carbon atoms. Examples of such groups include, but are not limited to, methyl, ethyl, propyl, iso-propyl, n-butyl, iso-butyl, tert-butyl, and such.

The term “C₂-C₁₀ alkenyl”, as used herein, unless otherwise indicated, includes alkyl moieties having at least one carbon-carbon double bond wherein alkyl is as defined above and including E and Z isomers of said alkenyl moiety, and having from 2 to 10 carbon atoms.

The term “C₂-C₁₀ alkynyl”, as used herein, unless otherwise indicated, includes alkyl moieties having at least one carbon-carbon triple bond wherein alkyl is as defined above, and containing from 2 to 10 carbon atoms.

A “C₃-C₁₀ cycloalkyl group” is intended to mean a saturated or partially saturated, monocyclic, or fused or spiro polycyclic, ring structure having a total of from 3 to 10 carbon ring atoms (but no heteroatoms). Exemplary cycloalkyls include cyclopropyl, cyclobutyl, cyclopentyl, cyclopentenyl, cyclohexyl, cycloheptyl, adamantyl, and like groups.

The term “C₆-C₁₀ aryl”, as used herein, unless otherwise indicated, includes an organic radical derived from an aromatic hydrocarbon by removal of one hydrogen, such as phenyl or naphthyl. The terms “Ph” and “phenyl,” as used herein, mean a —C₆H₅ group.

The term “4-10 membered heterocyclic”, as used herein, unless otherwise indicated, includes aromatic and non-aromatic heterocyclic groups containing one to four heteroatoms each selected from O, S and N, wherein each heterocyclic group has from 4-10 atoms in its ring system, and with the proviso that the ring of said group does not contain two adjacent O or S atoms. Furthermore, the sulfur atoms contained in such heterocyclic groups may be oxidized with one or two sulfur atoms. Non-aromatic heterocyclic groups include groups having only 4 atoms in their ring system, but aromatic heterocyclic groups must have at least 5 atoms in their ring system. The heterocyclic groups include benzo-fused ring systems. An example of a 4 membered heterocyclic group is azetidinyl (derived from azetidine). An example of a 5 membered heterocyclic group is thiazolyl and an example of a 10 membered heterocyclic group is quinolinyl. Examples of non-aromatic heterocyclic groups are pyrrolidinyl, tetrahydrofuranyl, dihydrofuranyl, tetrahydrothienyl, tetrahydropyranyl, dihydropyranyl, tetrahydrothiopyranyl, piperidino, morpholino, thiomorpholino, thioxanyl, piperazinyl, azetidinyl, oxetanyl, thietanyl, homopiperidinyl, oxepanyl, thiepanyl, oxazepinyl, diazepinyl, thiazepinyl, 1,2,3,6-tetrahydropyridinyl, 2-pyrrolinyl, 3-pyrrolinyl, indolinyl, 2H-pyranyl, 4H-pyranyl, dioxanyl, 1,3-dioxolanyl, pyrazolinyl, dithianyl, dithiolanyl, dihydropyranyl, dihydrothienyl, dihydrofuranyl, pyrazolidinyl, imidazolinyl, imidazolidinyl, 3-azabicyclo[3.1.0]hexanyl, 3-azabicyclo[4.1.0]heptanyl, 3H-indolyl and quinolizinyl. Examples of aromatic heterocyclic groups are pyridinyl, imidazolyl, pyrimidinyl, pyrazolyl, triazolyl, pyrazinyl, tetrazolyl, furyl, thienyl, isoxazolyl, thiazolyl, oxazolyl, isothiazolyl, pyrrolyl, quinolinyl, isoquinolinyl, indolyl, benzimidazolyl, benzofuranyl, cinnolinyl, indazolyl, indolizinyl, phthalazinyl, pyridazinyl, triazinyl, isoindolyl, pteridinyl, purinyl, oxadiazolyl, thiadiazolyl, furazanyl, benzofurazanyl, benzothiophenyl, benzothiazolyl, benzoxazolyl, quinazolinyl, quinoxalinyl, naphthyridinyl, and furopyridinyl. The foregoing groups, as derived from the groups listed above, may be C-attached or N-attached where such is possible. For instance, a group derived from pyrrole may be pyrrol-1-yl (N-attached) or pyrrol-3-yl (C-attached). Further, a group derived from imidazole may be imidazol-1-yl (N-attached) or imidazol-3-yl (C-attached). An example of a heterocyclic group wherein 2 ring carbon atoms are substituted with oxo (═O) moieties is 1,1-dioxo-thiomorpholinyl.

A “heteroaryl group” is intended to mean a monocyclic or fused or spiro polycyclic, aromatic ring structure having from 4 to 18 ring atoms, including from 1 to 5 heteroatoms selected from nitrogen, oxygen, and sulfur. Illustrative Examples of heteroaryl groups include pyrrolyl, thienyl, oxazolyl, pyrazolyl, thiazolyl, furyl, pyridinyl, pyrazinyl, triazolyl, tetrazolyl, indolyl, quinolinyl, quinoxalinyl, benzthiazolyl, benzodioxinyl, benzodioxolyl, benzooxazolyl, and the like.

The term “alkoxy”, as used herein, unless otherwise indicated, includes O-alkyl groups wherein alkyl is as defined above.

The terms “halogen” and “halo,” as used herein represent chlorine, fluorine, bromine or iodine.

The term “substituted,” means that the specified group or moiety bears one or more substituents. The term “unsubstituted,” means that the specified group bears no substituents. The term “optionally substituted” means that the specified group is unsubstituted or substituted by one or more substituents.

In accordance with a convention used in the art, the symbol 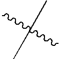
is used in structural formulas herein to depict the bond that is the point of attachment of the moiety or substituent to the core or backbone structure. In accordance with another convention, in some structural formulae herein the carbon atoms and their bound hydrogen atoms are not explicitly depicted, e.g., 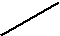
represents a methyl group, 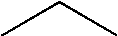
represents an ethyl group, 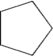
represents a cyclopentyl group, etc.

The term “stereoisomers” refers to compounds that have identical chemical constitution, but differ with regard to the arrangement of their atoms or groups in space. In particular, the term “enantiomers” refers to two stereoisomers of a compound that are non-superimposable mirror images of one another. The terms “racemic” or “racemic mixture,” as used herein, refer to a 1:1 mixture of enantiomers of a particular compound. The term “diastereomers”, on the other hand, refers to the relationship between a pair of stereoisomers that comprise two or more asymmetric centers and are not mirror images of one another.

The term “stereochemically-enriched” product, when used herein, refers to a reaction product wherein a particular stereoisomer is present in a statistically significant greater amount relative to the other possible stereoisomeric products. For example, a product that comprises more of one enantiomer than the other would constitute a stereochemically enriched product. Similarly, a product that comprises more of one diastereoisomer than others would also constitute a stereochemically enriched product. The methods and processes contained herein are said to afford a “stereochemically enriched ” product. In such cases, the methods and processes contained herein begin with a mixture of stereoisomeric compounds in which all possible stereoisomers are present in about an equal amount and afford a product in which at least one stereoisomer is present in a statistically significant greater amount than the others.

If the starting material constitutes a mixture of stereoisomers, such as a racemic mixture, one stereoisomer may react more slowly than the other in the presence of a chiral, non-racemic reagent or catalyst, such as a biocatalyst, an optically active base, or an optically active acid. Such a reaction may be referred to herein as a kinetic resolution, wherein the reactant enantiomers are resolved by differential reaction rates to yield both stereochemically-enriched product and stereochemically-enriched, unreacted starting material. Kinetic resolution is usually achieved by the use of a sufficient amount of a chiral, non-racemic reagent or catalyst to react with only one stereoisomer of the starting material.

The term “chiral, non-racemic base,” as used herein, means a basic compound that can exist in enantiomeric form and is not present in an equal amount with its correspondingly opposite enantiomer. For example, the compound 2-phenylglycinol exists as two enantiomers of opposite configuration, the so-called (R)— and (S)-enantiomers. If the (R)— and the (S)-enantiomers are present in equal amounts, such a mixture is said to be “racemic.” If, however, one enantiomer is present in an amount greater than the other, the mixture is said to be “non-racemic.”

The terms “resolution” and “resolving” mean a method of physically separating stereoisomeric compounds from a mixture of stereoisomers, such as a racemic mixture comprising two enantiomers of a particular compound. As used herein, “resolution” and “resolving” are meant to include both partial and complete resolution.

The terms “enzymatic process,” “enzymatic method,” “enzymatic reaction,” or “enzymatic resolution,” denote a process or method or reaction of the present invention employing an enzyme or microorganism.

The term “biocatalyst,” as used herein refers to an enzyme or mixture of enzymes that can be obtained from animals, plants, microorganisms, and the like. The enzyme or enzymes may be employed in any form such as in a purified form, a crude form, a mixture with other enzymes, a microbial fermentation broth, a fermentation broth, a microbial body, a filtrate of fermentation broth, and the like, either solely or in combination. In addition, the enzyme or microbial body may be immobilized, such as on a resin, or may be in solid form, such as in the form of a cross-linked enzyme crystal.

Furthermore, a “stereoselective process” is one that produces a particular stereoisomer of a reaction product in preference to other possible stereoisomers of that product. Enantioselectivity is generally quantified as “enantiomeric excess” (ee) defined as follows: [% enantiomeric excess A(ee)=(% enantiomer A)-(% enantiomer B)], where A and B are the enantiomeric products formed from the starting materials.

The compounds of the present invention may have asymmetric carbon atoms. The carbon-carbon bonds of the compounds of the present invention may be depicted herein using a solid line ( [symbol] ), a solid wedge 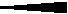
), or a dotted wedge 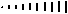
. The use of a solid line to depict bonds to asymmetric carbon atoms is meant to indicate that all possible stereoisomers at that carbon atom are included. The use of either a solid or dotted wedge to depict bonds to asymmetric carbon atoms is meant to indicate that only the stereoisomer shown is meant to be included. It is possible that compounds of the invention may contain more than one asymmetric carbon atom. In those compounds, the use of a solid line to depict bonds to asymmetric carbon atoms is meant to indicate that all possible stereoisomers are meant to be included. The use of a solid line to depict bonds to one or more asymmetric carbon atoms in a compound of the invention and the use of a solid or dotted wedge to depict bonds to other asymmetric carbon atoms in the same compound is meant to indicate that a mixture of diastereomers is present.

The term “treating,” as used herein, means allowing at least two chemical reactants to come into contact with one another such that a chemical reaction or transformation can take place. For example, in the processes of the present invention, a compound of formula (II) may be treated with a chiral, non-racemic base to afford a salt as a product of a chemical reaction. In such reactions, the compound of formula (II) is said to be treated with the base. Such reactions can occur in the solid phase, liquid phase, gas phase, in solution, or a combination of any of the foregoing depending on the identity of the reactants and their physical properties.

The terms “separating” or “separated,” as used herein, mean a process of physically isolating at least two different chemical compounds from each other. For example, if a chemical reaction takes place and produces at least two products, (A) and (B), the process of isolating both (A) and(B) in pure form is termed “separating” (A) and (B).

The term “hydrolyzed,” as used herein, means a chemical reaction, which may be mediated by a biocatalyst according to the present invention, in which an ester, an amide, or both are converted into their corresponding carboxylic acid derivatives. For example, if a reaction converts a compound of formula (I), wherein R⁶ is other than hydrogen to a compound of formula (I) wherein R⁶ is hydrogen, the compound of formula (I) is said to have been hydrolyzed.

The term “converting,” as used herein, means allowing a chemical reaction to take place with a starting material or materials to produce a different chemical product. For example, if chemical reactants (A) and (B) are allowed to react with each other to produce product (C), starting materials (A) and (B) can be said to have “converted” to product (C), or it can be said that (A) was “converted” to (C), or that (B) was “converted” to (C). Furthermore, in the processes of the invention, salt forms of compounds of formula (I) are said to be “converted” to a compound of formula (I). In such cases, the term “converted” means that the non-salt form of a compound of formula (I) was prepared from the corresponding salt form, usually by reaction with an appropriate acid, base, or combination of an acid and a base.

Solutions of individual stereoisomeric compounds of the present invention may rotate plane-polarized light. The use of either a “(+)” or “(−)” symbol in the name of a compound of the invention indicates that a solution of a particular stereoisomer rotates plane-polarized light in the (+) or (−) direction, as measured using techniques known to those of ordinary skill in the art.

### DETAILED DESCRIPTION

Diastereomeric mixtures can be separated into their individual diastereomers on the basis of their physical chemical differences by methods known to those skilled in the art, for example, by chromatography or fractional crystallization. Enantiomers can be separated by converting the enantiomeric mixtures into a diastereomeric mixture by reaction with an appropriate optically active compound (e.g., alcohol), separating the diastereomers and converting (e.g., hydrolyzing) the individual diastereomers to the corresponding pure enantiomers. All such isomers, including diastereomeric mixtures and pure enantiomers are considered as part of the invention.

Alternatively, individual stereoisomeric compounds of the present invention may be prepared in enantiomerically enriched form by asymmetric synthesis. Asymmetric synthesis may be performed using techniques known to those of skill in the art, such as the use of asymmetric starting materials that are commercially available or readily prepared using methods known to those of ordinary skill in the art, the use of asymmetric auxiliaries that may be removed at the completion of the synthesis, or the resolution of intermediate compounds using enzymatic methods. The choice of such a method will depend on factors that include, but are not limited to, the availability of starting materials, the relative efficiency of a method, and whether such methods are useful for the compounds of the invention containing particular functional groups. Such choices are within the knowledge of one of ordinary skill in the art.

When the compounds of the present invention contain asymmetric carbon atoms, the derivative salts, prodrugs and solvates may exist as single stereoisomers, racemates, and/or mixtures of enantiomers and/or diastereomers. All such single stereoisomers, racemates, and mixtures thereof are intended to be within the scope of the present invention.

As generally understood by those skilled in the art, an optically pure compound is one that is enantiomerically pure. Preferably, an optically pure compound according to the present invention comprises at least 90% of a single stereoisomer (80% enantiomeric excess), more preferably at least 95% (90% e.e.), even more preferably at least 97.5% (95% e.e.), and most preferably at least 99% (98% e.e.).

If a derivative used in the method of the invention is a base, a desired salt may be prepared by any suitable method known to the art, including treatment of the free base with an inorganic acid, such as hydrochloric acid; hydrobromic acid; sulfuric acid; nitric acid; phosphoric acid; and the like, or with an organic acid, such as acetic acid; maleic acid; succinic acid; mandelic acid; fumaric acid; malonic acid; pyruvic acid; oxalic acid; glycolic acid; salicylic acid; pyranosidyl acid, such as glucurotiic acid or galacturonic acid; alpha-hydroxy acid, such as citric acid or tartaric acid; amino acid, such as aspartic acid or glutamic acid; aromatic acid, such as benzoic acid or cinnamic acid; sulfonic acid, such as p-toluenesulfonic acid or ethanesulfonic acid; and the like.

If a derivative used in the method of the invention is an acid, a desired salt may be prepared by any suitable method known to the art, including treatment of the free acid with an inorganic or organic base, such as an amine (primary, secondary, or tertiary); an alkali metal or alkaline earth metal hydroxide; or the like. Illustrative Examples of suitable salts include organic salts derived from amino acids such as glycine and arginine; ammonia; primary, secondary, and tertiary amines; and cyclic amines, such as piperidine, morpholine, and piperazine; as well as inorganic salts derived from sodium, calcium, potassium, magnesium, manganese, iron, copper, zinc, aluminum, and lithium.

In the case of derivatives, prodrugs, salts, or solvates that are solids, it is understood by those skilled in the art that the derivatives, prodrugs, salts, and solvates used in the method of the invention, may exist in different polymorph or crystal forms, all of which are intended to be within the scope of the present invention and specified formulas. In addition, the derivative, salts, prodrugs and solvates used in the method of the invention may exist as tautomers, all of which are intended to be within the broad scope of the present invention.

The compounds of the present invention that are basic in nature are capable of forming a wide variety of different salts with various inorganic and organic acids. Although such salts must be pharmaceutically acceptable for administration to animals, it is often desirable in practice to initially isolate the compound of the present invention from the reaction mixture as a pharmaceutically unacceptable salt and then simply convert the latter back to the free base compound by treatment with an alkaline reagent and subsequently convert the latter free base to a pharmaceutically acceptable acid addition salt. The acid addition salts of the base compounds of this invention are readily prepared by treating the base compound with a substantially equivalent amount of the chosen mineral or organic acid in an aqueous solvent medium or in a suitable organic solvent, such as methanol or ethanol. Upon careful evaporation of the solvent, the desired solid salt is readily obtained. The desired acid salt can also be precipitated from a solution of the free base in an organic solvent by adding to the solution an appropriate mineral or organic acid.

Those compounds of the present invention that are acidic in nature are capable of forming base salts with various pharmacologically acceptable cations. Examples of such salts include the alkali metal or alkaline-earth metal salts and particularly, the sodium and potassium salts. These salts are all prepared by conventional techniques. The chemical bases which are used as reagents to prepare the pharmaceutically acceptable base salts of this invention are those which form non-toxic base salts with the acidic compounds of the present invention. Such non-toxic base salts include those derived from such pharmacologically acceptable cations as sodium, potassium, calcium and magnesium, etc. These salts can easily be prepared by treating the corresponding acidic compounds with an aqueous solution containing the desired pharmacologically acceptable cations, and then evaporating the resulting solution to dryness, preferably under reduced pressure. Alternatively, they may also be prepared by mixing lower alkanolic solutions of the acidic compounds and the desired alkali metal alkoxide together, and then evaporating the resulting solution to dryness in the same manner as before. In either case, stoichiometric quantities of reagents are preferably employed in order to ensure completeness of reaction and maximum yields of the desired final product.

The activities of the enzymes used in this invention are expressed in “units”. Units are defined as the rate of hydrolysis of p-nitrophenyl propionate per minutes as expressed in μmol/min at room temperature.

Specific examples of the enzymes that may be used according to the present invention are those obtained from animal and plants such as cow liver esterase, pig liver esterase, pig pancreas esterase, horse liver esterase, dog liver esterase, pig phosphatase, amylase obtainable from barley and potato and lipase obtainable from wheat. Other examples are hydrolases obtained from such microorganisms as Rhodotorula, Trichoderma, Candida, Hansenula, Pseudomonas, Bacillus, Achromobacter, Nocardia, Chromobacterium, Flavobacterium, Rhizopus, Mucor, Aspergillus, Alkaligenes, Pediococcus, Klebsiella, Geotrichum, Lactobaccilus, Cryptococcus, Pichia, Aureobasidium, Actinomucor, Enterobacter, Torulopsis, Corynebacterium, Endomyces, Saccaromyces, Arthrobacter, Metshnikowla, Pleurotus, Streptomyces, Proteus, Gliocladium, Acetobacter, Helminthosporium, Brevibacterium, Escherichia, Citrobacter, Absidia, Micrococcus, Microbacterium, Penicillium and Schizophyllium as well as from lichen and algae.

Specific examples of the microorganisms useful in the present invention include, but are not limited to, Rhodotorula minuta, Rhodotorula rubra, Candida krusei, Candida rugosa, Candida tropicalis, Candida utilus, Pseudomonas fragi, Pseudomonas putida, Pseudomonas fluorescens, Pseudomonas aeruginosa, Rhizopus chinensis, Mucor pusillus, Aspergillus niger, Alkaligenes faecalis, Torulopsis ernobii, Bacillus cereus, Bacillus subtilis, Bacillus pulmilus, Bacillus subtilis var. niger, Citrobacter freundii, Micrococcus varians, Micrococcus luteus, Pediococcus acidlactici, Klebsiella pneumoriae, Absidia hyalospora, Geotrichun candidum, Schizophyllum commune, Nocardia uniformis subtsuyanarenus, Nocardia uniformis, Chromobacterium chocolatum, Hansenula anomala var. ciferrii, Hansenula anomala, Hansenula polymorpha, Achromobacter lyticus, Achromobacter parvulus, Achromobacter sinplex, Torulopsis candida, Corynebacterium sepedonicum, Endomyces geotrichum, Saccaromyces carrvisial, Arthrobacter globiformis, Streptomyces grisens, Micrococcus luteus, Enterobacter cloacae, Corynebacterium ezui, Lacto bacillus casei, Cryptococcus albidus, Pichia polimorpha, Penicillium frezuentans, Aureobasidium pullulans, Actinomucor elegans, Streptomyces grisens, Proteus vulgaris, Gliocladium roseum, Gliocladium virens, Acetobacter aurantius, Helminthosporium sp. Chromobacterium iodinum, Chromobacterium violaceum, Flavobacterium lutescens, Metschnikowia pulcherrima, Pleurotus ostreatus, Brevibacterium ammoniagenes, Brevibacterium divaricatum, Escherichia coli, Rodotolura minuta var. texensis, Trichoderma longibrachiatum, Mucor javanicus, Flavobacterium arbonescens, Flavobacterium heparinum, and Flavobacterium capsulatum.

Exemplary, commercially available enzymes suitable for use in the present invention include lipases such as Amano PS-30 (Pseudomonas cepacla), Amano GC-20 (Geotrichum candidum), Amano APF (Aspergillus niger), Amano AK (Pseudomonas sp.), Pseudomonas fluorescens lipase (Biocatalyst Ltd.), Amano Lipase P30 (Pseudomonas sp.), Amano P (Pseudomonas fluorescens), Amano AY-30 (Candida rugosa), Amano N (Rhizopus niveus), Amano R (Penicillium sp.), Amano FAP (Rhizopus oryzae), Amano AP-12 (Aspergillus niger), Amano MAP (Mucor meihei), Amano GC-4 (Geotrichum candidum), Sigma L-0382 and L-3126 (porcine pancreas), Lipase OF (Sepracor), Esterase 30,000 (Gist-Brocades), KID Lipase (Gist-Brocades), Lipase R (Rhizopus sp., Amano), Sigma L-3001 (Wheat germ), Sigma L-1754 (Candida cytindracea), Sigma L-0763 (Chromobacterium viscosum) and Amano K-30 (Aspergillus niger). Additionally, exemplary enzymes derived from animal tissue include esterase from pig liver, chymotrypsin and pancreatin from pancreas such as Porcine Pancreatic Lipase (Sigma). Two or more, as well as a single, enzyme may be employed when carrying out the process of the present invention.

The buffer medium may be inorganic acid salt buffers (e.g. potassium dihydrogen phosphate, sodium dihydrogen phosphate), organic acid salt buffers (e.g. sodium citrate), or any other suitable buffer. The concentration of the buffer may vary from 0.005 to 2 M, preferably from 0.005 to 0.5 M and will depend on the specific subject compound and the enzymes or microorganism used.

A surfactant or mixture of surfactants may be added to the reaction mixture to solubilize the substrate. Examples of suitable surfactants include, but are not limited to, nonionic surfactants, such as alkylaryl polyether alcohols. One such surfactant that may be used is octylphenoxy polyethoxyethanol, commercially available as Triton X-100 (from Sigma Chemical Company). An effective amount of a surfactant is used. The amount used can vary from 0.05% to about 10%, depending on factors such as, but not limited to, the identity of the reactant or reactants, the identity of the product or products, the solvents and/or cosolvents used, and the preferred method of isolating the desired product or products. Whether such a surfactant or surfactants are necessary, the choice of a particular surfactant, and the amount used, are all choices within the knowledge of one of ordinary skill in the art and can be determined without undue experimentation.

An amount of an organic solvent or mixture of solvents may be added to the reaction mixture to increase reactant or product solubility to facilitate the reaction. Examples of suitable solvents include, but are not limited to, acetonitrile, tetrahydrofuran, dimethylsulfoxide, N,N-dimethylformamide, methyl alcohol, ethyl alcohol, and iso-propyl alcohol. Effective amounts of a co-solvent are from 1% to about 50% depending on the specific starting materials and enzymes and/or microorganism used. Whether such solvents are necessary, the identity of the solvent or solvents, and the amount of the solvent used are all choices within the knowledge of one of ordinary skill in the art and can be determined without undue experimentation.

The pH of the buffers or the pH of the reaction mixtures herein may be maintained from about 4 to about 10, from about 5 to about 9, or from about 7 to about 8. The reaction temperature may vary from about 0 to about 100° C., and will depend on the identity of the starting materials, the biocatalyst used, and the solvent or mixture of solvents used. The reaction time is generally from 1 hour to 400 hours and will depend on the identity of the starting materials, the biocatalyst used, and the solvent or mixture of solvents used. Reaction progress may be monitored by an appropriate analytical method, such as high-performance liquid chromatography (HPLC), reverse-phase HPLC, mass spectroscopy, proton nuclear magnetic resonance spectroscopy (NMR), or a combination of techniques, such as liquid chromatography/mass spectroscopy (LC/MS). The stereoselectivity of the reaction may be monitored or determined using techniques known to those of ordinary skill in the art, such as the use of HPLC with a chiral stationary phase. The conversion of starting materials may be carried to approximately 50%, after which the product acid and the unreacted starting material can be isolated.

The amount of enzyme used may vary from about 5 units to about 12,000 units of enzyme per mole of starting materials. The amount of a specific enzyme or mixture of enzymes required will depend on factors that include, but are not limited to, the temperature, the specific subject compound, the enzymes and/or microorganism used, and the desirable reaction time. It may also be desirable to use an excess of the enzymes or the enzymes in some cases to afford a practically short reaction time, especially when the enzymes are immobilized and can be reused for many turnovers. The concentration of the ester substrate may be from 0.1 g/L to 100 g/L and depends on the specific subject compound and the enzyme and/or microorganism used.

The enzymes and/or microorganisms used in the present invention may be in crude form or in an immobilized form. They can be immobilized on various solid supports without loss of stereospecificty or change in stereo selectivity. The solid supports can be inert absorbents to which the enzyme is not covalently bonded. Instead the enzyme is absorbed such as by interactions of hydrophobic or hydrophilic portions of a protein with like regions of the inert absorbent, by hydrogen bonding, by salt bridge formation, or by electrostatic interactions. Inert absorbent materials include, but are not limited to, synthetic polymers (e.g. polystyrene, poly-(vinylalcohol), polyethylene and polyamides), mineralaceous compounds (e.g. diatomaceous earth and Fuller'searth), or naturally occurring polymers (e.g. cellulose). Specific examples of such materials include Celite 545 diatomaceous earth, Abelite XAD-8 polymeric resin beads and polyethylene glycol 8000.

The enzyme may also be immobilized on the support to which the enzyme is covalently bonded (e.g., oxirane-acrylic beads and glutaraldehyde activated supports). Specific examples include Eupergit C oxirane-acrylic beads and glutaraldehyde activated Celite 545. Other possible immobilizing systems are well known and are readily available to those skilled in the art of enzyme immobilization.

The desired products, the optically pure (or enriched) unreacted ester and the optically pure (or enriched) acid may be isolated from the hydrolysis mixture using conventional methods such as extractions, acid-base extractions, filtration, chromatography, crystallization or combinations thereof. The recovered enzyme or microorganism may be recycled and used in subsequent reactions with or without further manipulation or purification.

Methods for separating final reaction products from each other and from the reaction components include, but are not limited to, filtration, distillation, liquid chromatography, column chromatography, sublimation, crystallization, and derivatization followed by any of the above methods. Which method is chosen to effect the desired separation will depend on factors that include, but are not limited to, the identity of the reaction components, starting materials, and products. These choices are within the knowledge of one of ordinary skill in the art and can be made without undue experimentation.

In a convenient isolation procedure, after the enzymatic hydrolysis, the pH is adjusted to pH 7.5 to 8 (in the case of immobilized biocatalysts, the biocatalyst is first separated by filtration), the product acid is separated from the unreacted ester by extracting the ester with an organic solvent such as methylene chloride, ethyl acetate, diethyl ether, methyl t-butyl ether, or any other solvent in which the substrate is soluble and stable. Concentration of the organic extracts affords the unreacted starting material. Concentration of the aqueous phase yields the product acid.

The acid can be freed of the buffer salts and enzyme by selective precipitation or chromatography or other methods known to those skilled in the art. These include acidifying the aqueous to about pH 3, or lower, and isolating the acid by extraction with an organic solvent such as methylene chloride, ethyl acetate, diethyl ether, methyl t-butyl ether, or any other solvent in which the acid is soluble and stable. Concentration of the organic extracts affords the unreacted starting material and the product acid, which can be purified and freed of the buffer salts and enzyme by selective precipitation or chromatography or other methods known to those of ordinary skill in the art.

Either the unreacted, stereoisomerically enriched starting material or the stereoisomerically enriched product can be further racemized, if so desired. The unreacted, stereoisomerically enriched starting material can be racemized by methods known to those of ordinary skill in the art, such as heating in the presence of a base and in the presence of an appropriate solvent or solvents. The stereoisomerically enriched product can be further racemized by converting it to an ester and heating in the presence of a base and in the presence of an appropriate solvent. The stereoisomerically enriched product can be converted to an ester using methods known to those of skill in the art, such as by heating the product in the presence of an alcohol and an appropriate acid. In this manner, any stereoisomer of the compounds of formulas (I) and (II) can be obtained in stereochemically enriched form.

The compounds of formula (II) can be prepared according to Scheme I shown below.

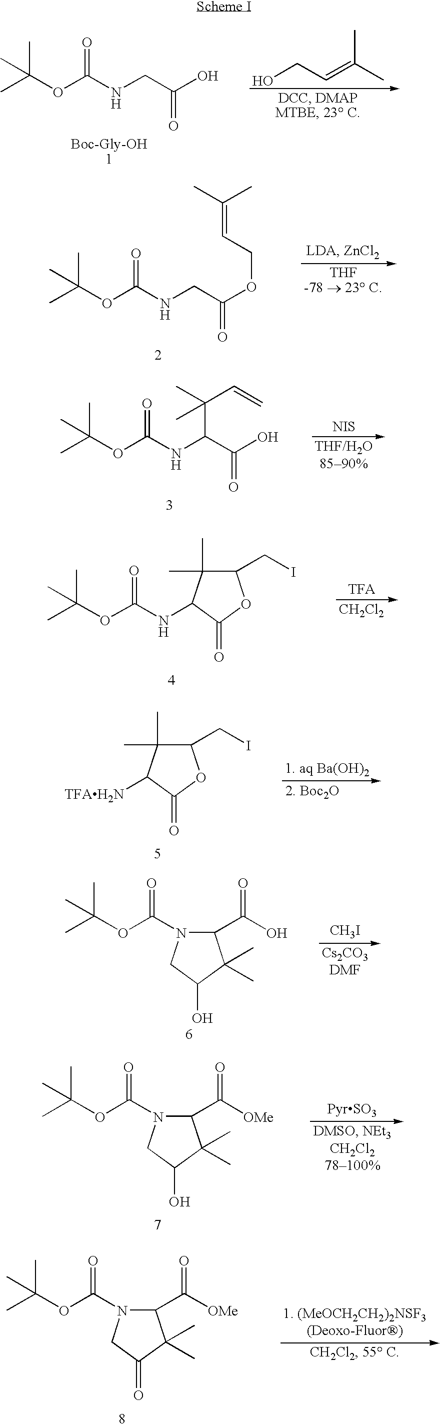

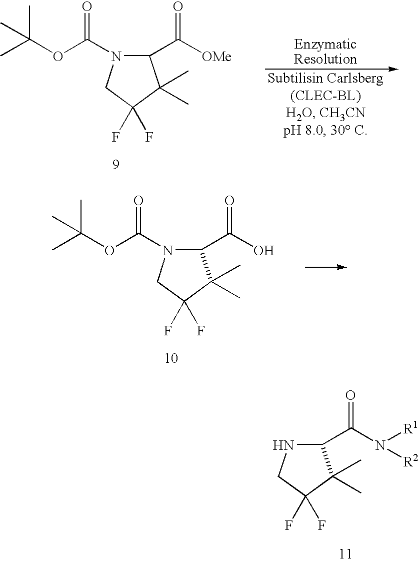

In general, one can begin with an N-protected glycine derivative, such as compound 1, which can be prepared from commercially available glycine according to methods known to those of ordinary skill in the art. The protected glycine derivative 1 can then be allowed to react with an agent or combination of agents that is capable of O-alkylating the terminal carboxyl group to afford compound 2. Examples of agents or combinations of agents that are known to O-alkylate a carboxyl group include, but are not limited to, alkyl halides, alkyl sulfonate esters, and alkyl trifluormethane sulfonate esters. These reactions may be performed in the presence of a base that will not interfere with the desired transformation. Such bases include, but are not limited to, inorganic bases such as sodium carbonate and potassium carbonate, and organic bases such as triethylamine or pyridine.

Alternatively, N-protected glycine derivatives such as compound 1 may be allowed to react with an alcohol in the presence of an agent or combination of agents that will convert the —OH group into a suitable leaving group. Examples of such agents or combination of such agents include, but are not limited to, dicyclohexylcarbodiimide, diisopropylcarbodiimide, 1-[3-(dimethylamino)propyl]-3-ethylcarbodiimide hydrochloride (EDC), 2-chloro-4,6-dimethoxy-1,3,5-triazine (CDMT), cyanuric chloride, 4-(4,6-dimethoxy-1,3,5-triazin-2-yl)-4-methylmorpholinium chloride, O-(7-azabenzotriazol-1-yl)-N,N,N′,N′-tetramethyluronium hexafluorophosphate (HATU), carbonyidiimidazole (CDI), benzotriazole-1-yl-oxy-tris-(dimethylamino)-phosphoniumhexafluorophosphate (BOP), 2-ethoxy-1-ethoxycarbonyl-1,2-dihydroquinoline (EEDQ), 2-(1H-benzotriazole-1-yl)-1,1,3,3-tetramethyluronium hexafluorophosphate (HBTU), 2-(1H-benzotriazole-1-yl)-1,1,3,3-tetramethyluronium tetrafluoroborate (TBTU), and 3-(diethoxyphosphoryloxy)-1,2,3-benzotriazin-4(3H)-one (DEPBT). These reactions may be performed in the presence of optional additives. Suitable additives include, but are not limited to, hydroxybenzotriazole (HOBt), hydroxyazabenzotriazole (HOAt), N-hydroxysuccinimide (HOSu), N-hydroxy-5-norbornene-endo-2,3-dicarboximide (HONB), and 4-dimethylaminopyridine (DMAP). Whether these additives are necessary depends on the identity of the reactants, the solvent, and the temperature, and such choices are within the knowledge of one of ordinary skill in the art.

In general, these reactions may be performed in a solvent that does not interfere with the reaction, for example alkyl or aryl ethers, alkyl or aryl esters, aromatic and aliphatic hydrocarbons, non-competitive alcohols, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C., depending on the specific reactants, solvents, and other optional additives used.

The O-alkylated glycine derivatives, such as compound 2, may also be prepared by reaction of the protected glycine derivatives with agents or a combination of agents that will convert the carboxylate into an acyl halide derivative, followed by reaction with an appropriate alcohol. For example, those compounds that contain an acyl chloride may be prepared from the protected glycine derivatives by reaction with agents such as thionyl chloride or oxalyl chloride. These reactions may be performed in the presence of a suitable base such as sodium carbonate, sodium bicarbonate, potassium carbonate, potassium bicarbonate, sodium hydroxide, potassium hydroxide, a trialkylamine, triethylamine for example, or a heteroaromatic base, pyridine for example. The resulting compounds may be isolated and then further reacted with an appropriate alcohol or they may be formed in situ and reacted with an appropriate alcohol without any isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

The O-alkylated glycine derivatives, such as compound 2, may also be prepared from the carboxylate by reaction with an agent or combination of agents that converts the carboxylate group into an acyl imidazole, followed by reaction with an appropriate alcohol. Suitable agents for converting the carboxylate to an acyl imidazole include, but are not limited to, carbonyl diimidazole. The acyl imidazole intermediates may be isolated and then further reacted with an appropriate alcohol or they may be formed in situ and reacted with an appropriate alcohol without isolation or further purification. These reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −20° C. to 100° C. The specific reaction conditions chosen will depend on the specific subject compound and reagents chosen. Such choices are within the knowledge of one of ordinary skill in the art.

The racemic compounds of formula (II), such as compound 3 shown in Scheme I, may be prepared from glycine derivatives that are O-alkylated with an allyl group or a derivative thereof, such as compound 2. Such O-alkylated glycine derivatives may be allowed to react with an agent or combination of agents such that the compound undergoes a Claisen-type rearrangement, to afford the compounds of formula (II). In general, these reactions may be performed in a solvent that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Additionally, water may be used as a co-solvent in this transformation if necessary. Furthermore, such reactions may be performed at temperatures from −78° C. to 100° C. The specific reaction conditions chosen will depend on the specific subject compound and reagents chosen. Such choices are within the knowledge of one of ordinary skill in the art.

In addition, such Claisen rearrangements may be promoted by first forming an enolate anion of the species that is to undergo the rearrangement. Such enolate anions can be prepared from the O-alkylated glycine derivatives by reaction with an agent or combination of agents that can function as a strong base. For example, the O-alkylated glycine derivatives can be allowed to react with lithium diisopropyl amide (LDA) to form the desired enolate anion. Such reactions may also be performed in the presence of additives that are known to promote such reactions, such as Lewis acids like zinc (II) chloride. In addition, such reactions can be performed in a solvent that will not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. Furthermore, such reactions may be performed at temperatures from −78° C. to 100° C. The specific reaction conditions chosen will depend on the specific subject compound and reagents chosen. Such choices are within the knowledge of one of ordinary skill in the art.

Compounds of formula (I), such as compound 10 as shown in Scheme I, may be prepared from compounds of formula (II). In general, the compound of formula (II) may be allowed to react with an electrophilic halogenating agent to afford a lactone, such as 4. Suitable electrophilic halogenating agents include, but are not limited to, N-chlorosuccinimide (NCS), N-bromosuccinimide (NBS), and N-iodosuccinimide (NIS). These reactions may be performed in a solvent or mixture of solvents that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. In addition, water may be added to the reaction mixtures if so desired. Furthermore, such reactions may be performed at temperatures from −78° C. to 100° C. The specific reaction conditions chosen will depend on the specific subject compound and reagents chosen. Such choices are within the knowledge of one of ordinary skill in the art.

The protected lactones, such as compound 4 in Scheme I, may be deprotected to provide alpha-amino lactones such as compound 5. Such deprotection reactions may be performed using methods known to those of ordinary skill in the art and as found in, for example, Greene et al., Protective Grouds in Organic Synthesis; John Wiley & Sons, New York, (1999).

The alpha-amino lactones, such as compound 5, may be allowed to react with an agent or combination of agents that allows the compound to undergo a rearrangement to afford a cyclic amine, such as compound 6 shown in Scheme I. In general, these reactions may be performed by allowing a compound such as 5 to react with an agent such as barium hydroxide. These reactions may be performed in a solvent or mixture of solvents that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. In addition, water may be added to the reaction mixtures if so desired. Furthermore, such reactions may be performed at temperatures from −78° C. to 100° C. The specific reaction conditions chosen will depend on the specific subject compound and reagents chosen. Such choices are within the knowledge of one of ordinary skill in the art.

The cyclic amines, such as compound 5 may be isolated as the free carboxy amine, or they may be derivatized to facilitate isolation. For example, as shown in Scheme I, compound 5 was allowed to react with barium hydroxide in a mixture of water and an organic solvent to afford the desired cyclic amine. The cyclic amine was then allowed to react with (BOC)₂O to afford the BOC-protected amine, compound 6.

Cyclic amines, such as compound 6, may then be allowed to react with an agent or combination of agents that is capable of O-alkylating the carboxylate group to afford an ester, such as compound 7. Such agents were described earlier and include, but are not limited to, methyl iodide, methyl sulfonate ester, and methyl bromide. Such reactions may be performed in the presence of a compound that is capable of acting as a base. Suitable bases include, but are not limited to, cesium carbonate, potassium carbonate, and sodium carbonate. In addition, these reactions may be performed in a solvent or mixture of solvents that does not interfere with the desired transformation. Among suitable solvents are alkyl or aryl ethers, alkyl or aryl esters, aromatic and aliphatic hydrocarbons, halogenated solvents, alkyl or aryl nitriles, alkyl or aryl ketones, aromatic hydrocarbons, or heteroaromatic hydrocarbons. For example, suitable solvents include, but are not limited to, ethyl acetate, isobutyl acetate, isopropyl acetate, n-butyl acetate, methyl isobutyl ketone, dimethoxyethane, diisopropyl ether, chlorobenzene, dimethyl formamide, dimethyl acetamide, propionitrile, butyronitrile, t-amyl alcohol, acetic acid, diethyl ether, methyl-t-butyl ether, diphenyl ether, methylphenyl ether, tetrahydrofuran, 2-methyltetrahydrofuran, 1,4-dioxane, pentane, hexane, heptane, methanol, ethanol, 1-propanol, 2-propanol, t-butanol, n-butanol, 2-butanol, dichloromethane, chloroform, 1,2-dichloroethane, acetonitrile, benzonitrile, acetone, 2-butanone, benzene, toluene, anisole, xylenes, and pyridine, or any mixture of the above solvents. In addition, water may be added to the reaction mixtures if so desired. Furthermore, such reactions may be performed at temperatures from −78° C. to 100° C. The specific reaction conditions chosen will depend on the specific subject compound and reagents chosen. Such choices are within the knowledge of one of ordinary skill in the art.

Compounds such as compound 7 may be resolved or prepared in stereochemically-enriched form using the methods of the present invention. Alternatively, compounds such as 7 may be used to prepare other compounds of formula (II) that may be resolved or prepared in stereochemically-enriched form according to the methods of the present invention. For example, the secondary hydroxyl group in compound 7 may be oxidized to afford the corresponding ketone 8, shown in Scheme I. Such oxidations may be performed by methods known to those of ordinary skill in the art, such as oxidation using PCC, under Swern conditions, or using pyr.SO_{3/}DMSO/NEt₃ Compounds such as 8 may be resolved or prepared in stereochemically-enriched form using the methods of the present invention. Alternatively, such compounds may be used to prepare other analogs that may themselves be resolved or prepared in stereochemically enriched form using the methods of the present invention.

For example, ketones such as compound 8 may be allowed to react with an agent or combination of agents that is capable of converting the ketone functional group into a dihalo methylene moiety, such as a —CF₂— group. Such reactions may be performed using agents or combinations of agents known to those of ordinary skill in the art, such as (diethylamino)sulfur trifluoride (DAST), and others. For example, reaction of compound 8 with (MeOCH₂CH₂)₂NSF₃ (sold as Deoxo-Fluor® by Air Products, Inc.) in dichloromethane and at 55° C., afforded the difluoro compound 9.

Compounds such as 9 can be resolved or prepared in stereochemically-enricned form using the methods of the present invention. As shown in Scheme I, reaction of a racemic mixture of compound 9 with the enzyme Subtilisin Carlsberg in a mixture of acetonitrile and water at a pH of 8, and at 30° C., provided stereochemically enriched compound 10. Compounds such as 10 that contain a nitrogen-protecting group can be further manipulated by removing the protecting group to afford secondary, cyclic amines such as compound 11 shown in Scheme I.

Alternatively, compounds such as 6, 7, 8, 9, and 10, as shown in Scheme I, can be prepared in stereochemically enriched form by resolving or preparing precursor compounds, such as compound 3 shown in Scheme I, in stereochemically enriched form. After resolving or preparing compounds such as 3 in stereochemically enriched form, they can be used as shown to prepare product compounds that are themselves stereochemically enriched.

Compounds of formula (I), wherein Z is O or S, can be prepared according to methods known to those of skill in the art. For example, see Mimoto, T. et al. J. Med. Chem. 1999, 42, 1789; EP 0751145; U.S. Pat. Nos. 5,644,028, 5,932,550, 5,962,640, 5,932,550, and 6,222,043, H. Hayashi et al., J. Med. Chem. 1999, 42, 1789; and PCT Publication No. WO 01/05230 A1, which are hereby incorporated by reference.

### EXAMPLES

The examples below are intended only to illustrate particular embodiments of the present invention and are not meant to limit the scope of the invention in any manner.

In the examples described below, unless otherwise indicated, all temperatures in the following description are in degrees Celsius (OC) and all parts and percentages are by weight, unless indicated otherwise.

Various starting materials and other reagents were purchased from commercial suppliers, such as Aldrich Chemical Company or Lancaster Synthesis Ltd., and used without further purification, unless otherwise indicated.

The reactions set forth below were performed under a positive pressure of nitrogen, argon or with a drying tube, at ambient temperature (unless otherwise stated), in anhydrous solvents. Analytical thin-layer chromatography was performed on glass-backed silica gel 60° F. 254 plates (Analtech (0.25 mm)) and eluted with the appropriate solvent ratios (v/v). The reactions were assayed by high-pressure liquid chromotagraphy (HPLC) or thin-layer chromatography (TLC) and terminated as judged by the consumption of starting material. The TLC plates were visualized by UV, phosphomolybdic acid stain, or iodine stain.

¹H-NMR spectra were recorded on a Bruker instrument operating at 300 MHz and ¹³CNMR spectra were recorded at 75 MHz. NMR spectra are obtained as DMSO-d₆ or CDCl₃ solutions (reported in ppm), using chloroform as the reference standard (7.25 ppm and 77.00 ppm) or DMSO-d₆ ((2.50 ppm and 39.52 ppm)). Other NMR solvents were used as needed. When peak multiplicities are reported, the following abbreviations are used: s=singlet, d=doublet, t=triplet, m=multiplet, br=broadened, dd=doublet of doublets, dt=doublet of triplets. Coupling constants, when given, are reported in Hertz.

Infrared spectra were recorded on a Perkin-Elmer FT-IR Spectrometer as neat oils, as KBr pellets, or as CDCl₃ solutions, and when reported are in wave numbers (cm⁻¹). The mass spectra were obtained using LC/MS or APCl. All melting points are uncorrected.

All final products had greater than 95% purity (by HPLC at wavelengths of 220 nm and 254 nm).

In the following examples and preparations, “Et” means ethyl, “Ac” means acetyl, “Me” means methyl, “Ph” means phenyl, (PhO)₂POCl means chlorodiphenylphosphate, “HCl” means hydrochloric acid, “EtOAc” means ethyl acetate, “Na₂CO₃” means sodium carbonate, “NaOH” means sodium hydroxide, “NaCl” means sodium chloride, “NEt3” means triethylamine, “THF” means tetrahydrofuran, “DlC” means diisopropylcarbodiimide, “HOBt” means hydroxy benzotriazole, “H₂O” means water, “NaHCO₃” means sodium hydrogen carbonate, “K₂CO₃” means potassium carbonate, “MeOH” means methanol, “i-PrOAc” means isopropyl acetate, “MgSO₄” means magnesium sulfate, “DMSO” means dimethylsulfoxide, “AcCl” means acetyl chloride, “CH₂Cl₂” means methylene chloride, “MTBE” means methyl t-butyl ether, “DMF” means dimethyl formamide, “SOCl₂” means thionyl chloride, “H₃PO₄” means phosphoric acid, “CH₃SO₃H” means methanesulfonic acid, “Ac₂O” means acetic anhydride, “CH₃CN” means acetonitrile, and “KOH” means potassium hydroxide.

### Example 1

### Preparation of (2S)-4,4-difluoro-3,3-dimethyl-N-Boc-proline

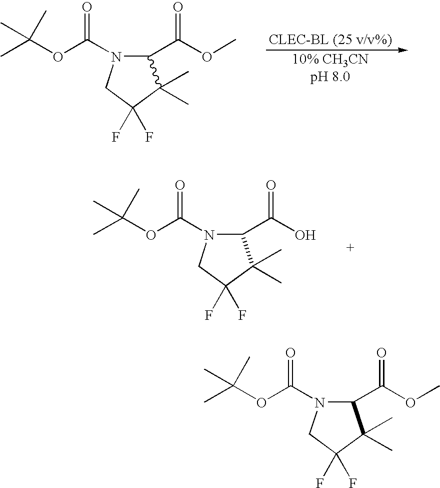

To a 50 L Reactor equipped with a pH electrode, an overhead stirrer, a heating coil and a base addition line (718 Stat Titrino-Metrohm pH titrator, Brinkman Instruments, Inc.), was added the alkaline protease from Bacillus licheniformis, (Subtilisin Carlsberg, purchased from Altus as CLEC-BL as a 6-14% w/v solution) (7 L of fresh CLEC+5L recycled CLEC (80 % of the initial activity) and 24 L of di-water). The pH of the suspension was adjusted to 8.0 by addition of 20 mL of 2N NaOH. A solution of the racemic ester (400 g, 1.36 mol, 1.00 eq, in acetonitrile, 3.6 L) was added to the suspension and the mixture was stirred at 30° C. for 262 h. During the reaction time, the pH of the solution was monitored and was maintained at pH 8.0 by the periodic addition of 2N NaOH (a total of 246 mL of base were added during the 262 h reaction time). Reaction progress was monitored using reverse-phase HPLC. The reaction was stopped after it had been determined that 45-50% starting material had been consumed. The enantiomeric excess (% ee) of the acid was determined to be 95.5%. The reaction mixture was extracted MTBE (3× with 16 L), and the combined organic layers were dried over Na₂SO₄ and concentrated under vacuum to afford 220 g of crude scalemic ester 1, (R)-enriched. The remaining aqueous slurry was filtered (to remove the CLEC-BL) through Whatman paper 1. The CLEC paste was removed from the paper and stored at 4° C. for later use, if desired. The remaining aqueous solution was acidified to pH 5.5 with 1 N hydrochloric acid and extracted with MTBE (2×16 L each). The aqueous solution was again extracted, at pH 5.0 and at pH 4.0. The organic fractions containing product acid were pooled, and concentrated with vacuum to afford a solid residue. The residue was suspended in hot water (40-50° C., 1000 mL) and allowed to cool to room temperature overnight. The resulting slurry was filtered and the crystals dried in a vacuum oven at 40° C. overnight to afford the acid as a white solid (133 g, 98% ee, 69.8% yield for kinetic resolution, >98% HPLC pure). ¹H NMR (300 MHz, CDCl3): δ 7.9 (bs,1 H), 4.10 (d,1 H), 3.89 (dd, 2H), 1.5 (s)+1.45 (s) (9H), 1.3 (s,3H), 1.15 (s, 3H).

The following analytical methods were used to monitor reaction progress and measure the % ee and purity of the final product:

Non-Chiral HPLC:

Detector wavelength: 200 nm
Column: Luna C-18, 4.6×30 mm
Column temperature: 35° C.
Flow rate 1.5 mL/min
Injection volume: 10 μL
Mobile Phases: A: 25 mM KH₂PO₄ pH 2.5; B: Acetonitrile
Run: gradient: 35 to 70% B in 5 min, 2 min post run
Retention times: Acid 1.63; Ester 3.28
   Chiral HPLC:
Detector wavelength: 195 nm
Column: Chiralcel OJ-R, 3 μm, C-18, 4.6×150 mm
Column temperature: 40° C.
Flow rate 0.5 mL/min
Injection volume: 10 μL
Mobile Phases: A: 25 mM KH₂PO₄ pH 2.0; B: Acetonitrile; C: HPLC H₂O
Run: Isocratic: 75% A and 25% B for 17 min, then 75% B and 25% C for 3 min, and finally, 75% A and 25% B for 15 min
Retention times: Acid 14.85(R) and 15.84 (S)
   Sample Preparation

Two samples were taken each time sampling was performed. Every sample was prepared by mixing 10 mL from the reaction mixture and 10 mL acetonitrile. The solution was vortexed and layers were separated by centrifugation on a Beckman microfuge. 100 μL of the upper layer were further diluted with 400 μL of acetonitrile and injected into the HPLC.

### Example 2

### Preparation of (2S)-4,4-difluoro-3,3-dimethyl-N-Boc-proline

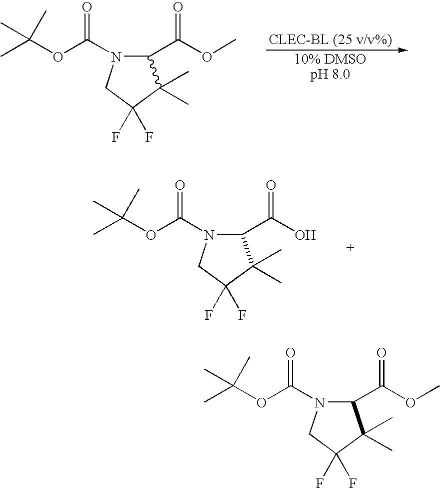

A solution of racemic ester (3 g, 10.23 mmol, 1.00 eq in DMSO, 15 mL) and 50 mM TRIS buffer at pH 8 (97.5 mL) were added to a 250 mL 3-neck flask equipped with a temperature probe, a stirring bar, a pH electrode and a base addition line. The pH of the mixture was approximately 7.64. The alkaline protease from Bacillus licheniformis, (Subtilisin Carlsberg, purchased from Altus as CLEC-BL as a 6-14% w/v solution) (37.5 mL) was then added. After addition of the enzyme, the pH of the mixture was approximately 7.70. The resulting mixture was slowly heated to 40° C. using a heating mantle. The pH of the mixture was adjusted to pH 8.0 by the addition of 1 N sodium hydroxide (0.848 mL). As the reaction progressed, the pH of the reaction mixture was monitored and maintained at pH 8 by the periodic addition of 1 N sodium hydroxide (a total of 17.7 mL of 1N NaOH were added). The resulting suspension was stirred at 40° C. for a total of 121 h. Reaction progress was monitored by reverse-phase HPLC monitoring both conversion and % ee of the product. The reaction was stopped after HPLC analysis indicated that 43% of the starting material had been consumed. The % ee of the acid was measured as 94.6% ee.

The resulting mixture was extracted MTBE (three ×75 mL each) and the combined organic layers were filtered through Whatman paper number 1 to remove emulsified particulates and to better distinguish the aqueous-organic boundary. The aqueous layer associated with the emulsion was added back to the mother liquor. The organic layer was dried over Na₂SO₄ and concentrated under vacuum to afford 1.74 g of crude scalemic ester I, (R)-enriched.

The remaining aqueous slurry was filtered (to remove the CLEC-BL) through Whatman paper 1. The CLEC-BL paste was removed from the paper and stored at 4° C. for later use, if desired. The remaining aqueous solution was acidified to pH 5.3 using 1 N HCl and extracted with 80 mL MTBE. The extraction was repeated five times with the pH reduced to 5.3, 4.8, 4.0, and 3.9, respectively in the subsequent four extractions. The organic layers containing acid were pooled, dried over Na₂SO₄, and concentrated with vacuum to obtain 1.460 g of crude acid. The solid residue was then resuspended in 150 mL MTBE. The acid was washed twice with 75 mL of 10 mM potassium phosphate buffer at pH 4.2-4.5 to remove DMSO. The organic layer was dried with Na₂SO₄ and concentrated under vacuum to afford the acid as a white solid (0.831 g, 98% ee, 58% yield for kinetic resolution, >98% HPLC pure). ¹H NMR (300 MHz, CDCl3): δ 5.3 (bs, 1H),4.15 (d, 1H), 3.89 (dd, 2H 1.5 (s)+1.45 (s, 9H), 1.3 (s, 3H) 1.15 s, 3H).

The following analytical methods were used to monitor reaction progress and measure the % ee and purity of the final product:

Non-Chiral HPLC:

Detector wavelength: 200 nm
Column: Luna C-18, 4.6×30 mm
Column temperature: 35° C.
Flow rate 1.5 mL/min
Injection volume: 10 μL
Mobile Phases: A: 25 mM KH₂PO₄ pH 2.5; B: Acetonitrile
Run: gradient: 35 to 70% B in 5 min, 2 min post run
Retention times: Acid 1.63; Ester 3.28
   Chiral HPLC:
Detector wavelength: 195 nm
Column: Chiralcel OJ-R, 3 □m, C-184.6×150 mm
Column temperature: 40° C.
Flow rate 0.5 mL/min
Injection volume: 10 □L
Mobile Phases: A: 25 mM KH₂PO₄ pH 2.0; B: Acetonitrile; C: HPLC H₂O
Run: Isocratic: 75% A and 25% B for 17 min, then 75% B and 25% C for 3 min, and finally,
75% A and 25% B for 15 min
Retention times: Acid 14.85(R) and 15.84 (S)
   Sample Preparation:

Two samples were taken each time sampling was performed. Each sample was prepared by diluting 100 μL of the reaction mixture with 1.9 mL acetonitrile. The solution was vortexed and 1 mL of this solution was centrifuged on a Beckman microfuge. The upper layer was injected into HPLC.

### Example 3

### Resolution of N-benzyl-4,4-difluoro-3,3-dimethylproline methyl ester

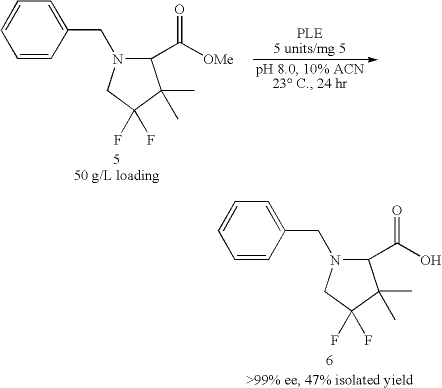

To N-tert-butoxycarbonyl-4,4-difluoro-3,3-dimethylproline methyl ester (5.0 g, 17.0 mmol) was added 4M HCl (2.0 eqv, 8.5 mL) in dioxane. The mixture was stirred at 0° C. for 3 h. After the reaction was judged to be complete by HPLC, the dioxane and excess HCl were removed in vacuo to afford a crude residue. The crude residue was washed by MTBE (3 mL×2) and dried in oven to provide 3.8 g solid 4,4-difluoro-3,3-dimethylproline methyl ester (˜95%purity), which was used in the next reaction without any further purification.

To 3.8 g of 4,4-difluoro-3,3-dimethylproline methyl ester was added K₂CO₃ (3.8 g), MeOH (60 mL), and BnBr (1.1 eqv, 2.55 mL). The resulting mixture was stirred at 23° C. for 20 h. The mixture was filtered and the solvents were removed under vacuum, leaving a residue. The residue was dissolved in MTBE (60 mL) and was washed with 1N HCl (20 mL×3), NaHCO₃(20 mL×3) and brine (20 mL×1). The organic layers were combined, dried over Na₂SO4, and the solvents were removed under vacuum to afford 4.3 g (90% yield) of N-benzyl-4,4-difluoro-3,3-dimethylproline methyl ester.

The racemic ester (3.0 g, 10.6 mmol) was dissolved in acetonitrile (9 mL, 15%) and potassium phosphate buffer (pH 8.0, 0.1 M, 60 mL) was added. Pig Liver esterase (750 mg, 10-15 units/mg) was added and the pH of the solution was maintained at pH 8 by the periodic addition of 1N NaOH.

Reaction progress was monitored by reverse-phase HPLC. After 20-24 h, ˜50% conversion was reached and the pH of the mixture was adjusted to pH 8.3-8.4 by the addition of 1N NaOH and the solution was extracted with MTBE (40 mL×3). The organic layer was dried over Na₂SO₄ and was concentrated to afford the remaining R-ester 5 (1.56 g, ˜52%). The pH of the remaining aqueous layer was adjusted to pH 3.5 by the addition of 1N HCl and was extracted with MTBE (40 mL×3). The combined organic layers were dried over Na₂SO₄, and the solvents were removed under vacuum to afford N-benzyl-4,4-difluoro-3,3-dimethylproline (1.35 g, 47%).

4,4-difluoro-3,3-dimethylproline methyl ester: ESI [M+H]⁺ 194.1. ¹H NMR (300 MHz, D₂O): δ 4.48 (s, 1H), 3.77-3.98 (m, 2H), 3.80 (s, 3H), 1.30 (s, 3H), 1.04 (s, 3H). ¹³C NMR (75 ppm, D₂O) δ167.70, 66.43, 66.38, 54.52, 48.59, 45.94, 17.64, 17.02.

N-benzyl-4,4-difluoro-3,3-dimethylproline methyl ester: ESI [M+H]⁺ 284.1. ¹H NMR (300 MHz, CDCl₃): δ 7.23-7.40 (m, 5H), 3.94 (d, J=13.2 Hz, 1H), 3.48 (d, J=13.2 Hz, 1H), 3.39 (s, 1H), 3.39 (s, 1H), 3.35 (dd, J=10.6, 20.0 Hz, 1H), 2.85 (ddd, J=6.6, 11.4, 18.3 Hz, 1H), 1.22 (s, 3H), 1.07 (s, 3H). ¹³C NMR (75 ppm, CDCl₃) δ171.42, 138.20, 128.87, 128.73, 127.73, 74.18, 58.72, 51.98, 47.06, 33.82, 20.16, 18.96.

N-benzyl-4,4-difluoro-3,3-dimethylproline: ESI [M-H]⁻ 268.1. ¹H NMR (300 MHz, CDCl₃): δ 7.26-7.40 (m, 5H), 3.94 (d, J=12.9 Hz, 1H), 3.66 (d, J=12.9 Hz, 3.32-3.57 (m, 2H), 3.05 (m, 1H), 1.26 (s, 3H), 1.11 (s, 3H). ¹³C NMR (75 ppm, CDCl₃) δ171.10, 136.19, 129.16, 128.68, 127.73, 74.83, 60.67, 56.49, 53.75, 46.48, 20.40, 18.77.

The following analytical methods were used:
N-benzyl-4,4-difluoro-3,3-dimethylproline methyl ester: Chiralcel AD-RH (4.6×100 mm, 3 μm); flow rate: 0.6 mL/min; injection volume: 5 μL; mobile phases: ACN/H₂O (20:80), detection at 254 nm.
N-benzyl-4,4-difluoro-3,3-dimethylproline: Chiralcel OD-RH (4.6×100 mm, 3 μm); flow rate: 0.6 mL/min; injection volume: 5 μL; mobile phases: ACN/H₂O (60:40), detection at 254 nm.

### Example 4

### Preparation of (S)-3,3-dimethyl-N-Boc-vinylglycine

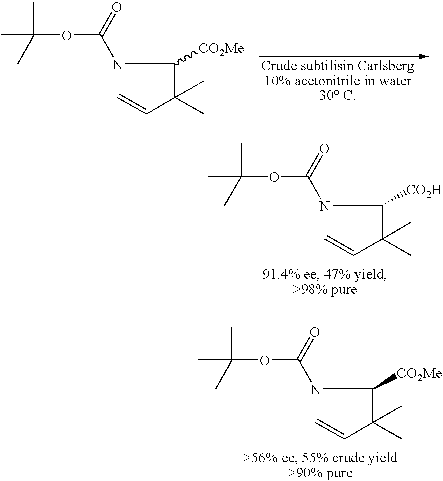

To a 5 L three neck flask equipped with a pH electrode, an overhead stirrer, a heating mantle and a base addition line, was added the racemic ester I (78 g, 0.3 mol, 1.00 eq) in acetonitrile (280 mL).

In a separate container were added Alcalase (350 mL of a solution that was passed through a tangential filtration system and concentrated to one fifth of the original volume) and distilled water (2.8 L). The pH of the resulting solution was adjusted to pH 7.0. The enzyme solution was added to flask containing the ester solution. The resulting suspension was stirred at 30° C. for 51 h, during which time the pH of the solution was maintained at 7.0 by the periodic addition of 1N NaOH (a total of 95.8 mL of base added over the 51 h). Reaction progress was followed by reverse-phase HPLC and the reaction was stopped after it was determined that 45% of the starting material had been consumed.

The mixture was extracted with MTBE (3×1.75 L each), and the combined organic layers were dried over MgSO₄ and concentrated under vacuum to afford 50.81 g of crude scalemic ester I, (R)-enriched (>55% yield, approx. 56% ee). This crude mixture contained some carboxylic acid <7%, which was recovered later by acid-base extraction. The remaining aqueous solution was passed through a Pellicon 2 tangential flow filtration equipped with an Ultracel cellulose membrane. The remaining solution was acidified to pH 4.0 and extracted with MTBE (3×1.75 L). The fractions containing the acid were pooled, dried over sodium sulfate and concentrated under vacuum to afford a pale yellow oil (31 g, 91.4% ee, 42% yield, >98% HPLC pure). ¹H NMR (300 MHz, CDCl3): δ 10.69 (s, 1H), 5.78 (dd, 2H), 5.02 (m, 2H), 4.96 (s,1 H), 4.09 (d, 1 H), 1.36 (s, 9H), 1.06 (s, 6H).

The following analytical conditions were used:

Non-Chiral HPLC

Detector wavelength: 200 nm
Column: Luna C-18, 4.6×30 mm
Column temperature: 35° C.
Flow rate 1.5 mL/min
Injection volume: 10 μL
Mobil Phases: A: 25 mM KH₂PO₄ pH 2.5; B: Acetonitrile
Run: gradient: 30 to 70% B in 5 min, 2 min pre run
Retention times: Acid 1.63; Ester 3.28
   Chiral HPLC
Detector wavelength: 200 nm
Column: Chiralcel OJ-R, 3 μm, C-18, 4.6×100 mm
Column temperature: not controlled
Flow rate 0.5 mL/min
Injection volume: 10 μL
Mobil Phases: A: 25 mM NaH₂PO₄ pH 2.0; B: Acetonitrile
Run: Isocratic: 25% B for 55 min, 3 min post run
Retention times: Acid 16.33 (R) and 17.97 (S); Ester 50.40 (S), 51.30(R)
Method to analyze ee of pure ester:
Column: Chiralcel OD-RH, 150×4.6 mm
Flow rate: 0.8 mL/min
Temperature: 30° C. degree
Mobile phrase: 30% ACN and 70% H₂O
Wavelength: 205 nm
Sample preparation:
   Sample Preparation:

Two samples were taken each time sampling was performed. Every sample was made by taking 2×200 μL from the reaction mixture, diluted with 1 mL of ethyl acetate and 100 □L of 1N HCl, then vortexed and layers separated by centrifugation on a Beckmann microfuge. 100 □L of the upper layer (organic) were further diluted with 400 μL of acetonitrile/water (1:1) and injected into HPLC.

### Example 5

### Preparation of (R)-3,3-dimethyl-N-Boc-vinylglycine

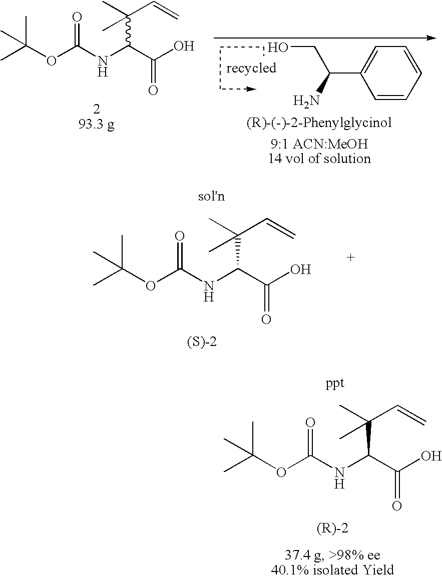

To a 2000 mL jacketed flask equipped with an overhead stirrer was added the racemic acid 2, 3,3-dimethyl-N-Boc-vinylglycine, (93.3 g, 386.2 mmol, 1.00 eq), (R)-phenylglycinol (52.6 g, 386.2 mmol, 1.00 eq), methanol (200 mL) and acetonitrile (1800 mL). The resulting slurry was stirred and heated to 70-80° C. until the solution became homogeneous. The solution was allowed to slowly cool to room temperature with continuous stirring, resulting in crystallization. The resulting slurry was filtered and the crystalline salt (containing the desired (R)-enantiomer) was washed with 100 mL of cold acetonitrile, collected and analyzed by HPLC. In cases where it is desired to improve the % ee of the resulting product after the 1^{st} crystallization, a second crystallization can be performed.

The salt was then converted to the free acid by dissolving the salt in 250 mL of ethyl acetate (alternatively, MTBE can be used in place of ethyl acetate). Water (250 mL) was then added and the pH of the resulting solution was adjusted to pH 3 by the addition of 1N hydrochloric acid. The organic layer was separated and the aqueous phase was again extracted with ethyl acetate (200 mL). The extracts were combined, dried over sodium sulfate, and concentrated under vacuum to afford a clear oil that was dried overnight under vacuum to afford a white solid (37.4 g, >98% ee, 40.1% isolated overall yield, >98% pure).

Recvcling (R)-Phenylglycinol:

The pH of the aqueous layer from the previous step was adjusted to pH 8.0 by the addition of 1N sodium hydroxide and the solution was extracted with 300 mL of Ethyl Acetate (or MTBE). The extract was then dried with sodium sulfate and concentrated under vacuum. The product was isolated as white crystals (12.3 g, >98% pure, note that recovery was only from 40% of material, the remaining recrystallizing agent can be recovered from the filtrate generated in step 4).

The following analytical methods were used:

Chiral HPLC Conditions

Detector wavelength: 205 nm
Column: Chiralcel OJ-RH, 3μ, C-184.6×100 mm
Column temperature: 30° C.
Flow rate 0.6 mL/min
Injection volume: 10 μL
Mobil Phases: A: Acetonitrile (0.1% TFA): B: 75% H₂O (0.1% TFA)
Run: Isocratic: 25% A: 75% B 18 min
Retention times: Acid: 11.69 (R) and 12.9 (S)/5.3 R-phenyl glycinol

### Example 6

### Preparation of (2S)-4-oxo-3,3-dimethyl-N-Boc-proline

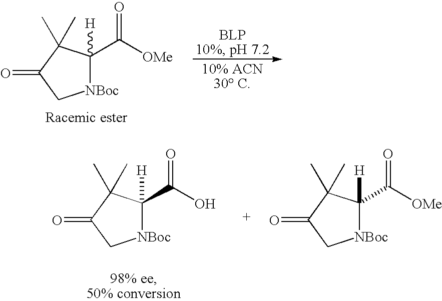

10 mg of the racemic ester was suspended in 100 microliters of acetonitrile and was added to 900 mL of KPB buffer containing 10% Bacillus lentus protease (BLP). The reaction was stopped after 16 h, resulting in a 50% conversion and >98% ee of the acid product (2S)-4-oxo-3,3-dimethyl-N-Boc-proline. Amano proleather FGF was another candidate, which was found to carry out this reaction with similar enatioselectivities and reactivity.

1. A method of preparing a stereoisomerically enriched compound of formula (I), 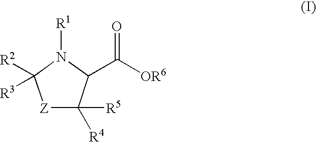
wherein:
Z is O, S, C═O, C═CH₂, or —(CR⁷R⁸)—;
R¹ is hydrogen, —(CR⁷R⁸)ₜ(C₆-C₁₄ aryl), —CH₂CH═CH₂, —C(O)R⁷, —C(O)OR⁷, —C(O)C(O)OR⁷, or —Si(R⁷)₃, wherein said C₆-C₁₄ aryl is optionally substituted with at leas substituent chosen from halo, C₁-C₁₀ alkyl, —OR⁷, and —N(R⁷R⁸);
R² and R³ are independently chosen from hydrogen, C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁷, and —N(R⁷R⁸);
R⁴ and R⁵ are independently chosen from hydrogen, halo, C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁷, and —N(R⁷R⁸);
R⁶ is hydrogen;
each R⁷ and R⁸ is independently chosen from hydrogen, halo, C₁-C₁₀ alkyl, C₁-C₁₀ alkoxy, C₂-C₁₀ alkenyl, C₂-C₁₀ alkynyl, —(CR⁹R⁹)ₜ(C₆-C₁₄ aryl), and —(CR⁹R⁹)ₜ(4-10 membered heterocyclic), wherein said C₆-C₁₄ aryl and 4-10 membered heterocyclic are optionally substituted with at least one substituent chosen from halo, C₁-C₁₀ alkyl, —OR⁹, and —N(R⁹R⁹);
each R⁹ is independently chosen from hydrogen and C₁-C₁₀ alkyl; and
t is an integer from 0 to 5;
said method comprising:
treating a compound of formula (I), wherein R¹, R², R³, R⁴ and R⁵ are as defined above and R⁶ is chosen from C₁-C₁₀ alkyl, C₂-C₁₀ alkenyl, C₂-C₁₀ alkynyl, —(CR⁷R⁸)ₜ(C₆-C₁₄ aryl), and —(CR⁷R⁸)ₜ(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁸), with a biocatalyst in an aqueous solution, an organic solvent, or a mixture of organic and aqueous solvents wherein at least one stereoisomer is selectively hydrolyzed.

2. A method according to claim 1, wherein in the compound of formula (I):
Z is O, S, C═O, C═CH₂, or —(CR⁷R⁸)—;
R¹ is —(CH₂)(C₆-C₁₄ aryl), —CH₂CH═CH₂, —C(O)OR⁷, or —C(O)C(O)OR⁷, wherein said C₆-C₁₄ aryl is optionally substituted with at least one substituent chosen from halo, C₁-C₁₀ alkyl, —OR⁷, and —N(R⁷R⁸);
R² and R³ are independently chosen from hydrogen, methyl, ethyl, butyl, and pentyl;
R⁴ and R⁵ are independently chosen from hydrogen, halo, methyl, ethyl, butyl, and pentyl;
R⁶ is hydrogen;
R⁷ and R⁸ are independently chosen from hydrogen, halo, C₁-C₁₀ alkyl, C₁-C₁₀ alkoxy, C₂-C₁₀ alkenyl, and C₆-C₁₄ aryl, wherein said C₆-C₁₄ aryl is optionally substituted with at least one substituent chosen from halo, C₁-C₁₀ alkyl, —OR⁹, and —N(R⁹R⁹); and
each R⁹ is independently chosen from hydrogen and C₁-C₁₀ alkyl.

3. A method according to claim 1, wherein in the compound of formula (I):
Z is O, S, C═O, C═CH₂, or —(CR⁷R⁸)—;
R¹ is —CH₂Ph, —C(O)OR⁷, or —C(O)C(O)OR⁷;
R² and R³ are hydrogen;
R⁴ and R⁵ are independently chosen from hydrogen and methyl;
R⁶ is hydrogen; and
R⁷ and R⁸ are independently chosen from hydrogen and C₁-C₁₀ alkyl.

4. A method according to claim 1, wherein in the compound of formula (I):
Z is O, S, C═O, C═CH₂, or —(CR⁷R⁸)—;
R¹ is —CH₂Ph, —C(O)OCH₃, —C(O)OC(CH₃)₃, or —C(O)C(O)OCH₃;
R² and R³ are hydrogen;
R⁴ and R⁵ are independently chosen from hydrogen and methyl;
R⁶ is hydrogen; and
R⁷ and R⁸ are independently chosen from hydrogen, fluorine, methyl, and —OCH₃.

5. A method according to claim 1, wherein in the compound of formula (I):
Z is S;
R¹ is —CH₂Ph, —C(O)OCH₃, —C(O)OC(CH₃)₃, or —C(O)C(O)OCH₃;
R² and R³ are hydrogen;
R⁴ and R⁵ are methyl; and
R⁶ is hydrogen.

6. A method according to claim 1, wherein in the compound of formula (I):
Z is —(CR⁷R⁸)—;
R¹ is —CH₂Ph, —CH₂CH═CH₂, —C(O)OCH₃, —C(O)OC(CH₃)₃, or —C(O)C(O)OCH₃;
R² and R³ are hydrogen;
R⁴ and R⁵ are independently chosen from hydrogen and methyl, ethyl, butyl and pentyl;
R⁶ is hydrogen; and
R⁷ and R⁸ are independently chosen from hydrogen, fluorine, chlorine, C₁-C₁₀ alkyl, and C₁-C₁₀ alkoxy.

7. A method according to claim 1, wherein said biocatalyst is chosen from an alkaline protease, an esterase, a lipase, a hydrolase, and any combination thereof.

8. A method of preparing a stereoisomerically enriched compound of formula (IA), 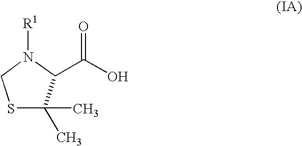
wherein:
R¹ is —CH₂Ph, —C(O)OR⁷, or —C(O)C(O)OR⁷; and
R⁷ is C₁-C₁₀ alkyl;
said method comprising:
treating a compound of formula (IC), 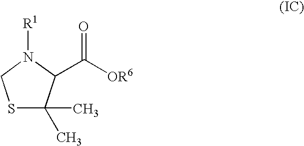
wherein R¹ is as defined above and R⁶ is chosen from C₁-C₁₀ alkyl, C₂-C₁₀ alkenyl, C₂-C₁₀ alkynyl, —CH₂(C₆-C₁₄ aryl), and —CH₂(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁷), with a biocatalyst in an aqueous solution, an organic solvent, or a mixture of organic and aqueous solvents wherein at least one stereoisomer is selectively hydrolyzed.

9. A method according to claim 8, wherein said biocatalyst is chosen from an alkaline protease, an esterase, a lipase, a hydrolase, and any combination thereof.

10. A method of preparing a stereoisomerically enriched compound of formula (IB), 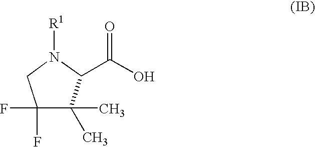
wherein:
R¹ is —CH₂Ph, —C(O)OR⁷, or —C(O)C(O)OR⁷; and
R⁷ is C₁-C₁₀ alkyl;
said method comprising:
treating a compound of formula (ID), 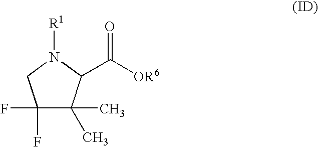
wherein R¹ is as defined above and R⁶ is chosen from C₁-C₁₀ alkyl, C₂-C₁₀ alkenyl, C₂-C₁₀ alkynyl, —CH₂(C₆-C₁₄ aryl), and —CH₂(4-10 membered heterocyclic), and wherein said C₆-C₁₄ aryl and 4-10 membered heterocyclic are optionally substituted with at least one substituent chosen from halo, C₁-C₁₀ alkyl, —OR⁷, and —N(R⁷R⁷), with a biocatalyst in an aqueous solution, an organic solvent, or a mixture of organic and aqueous solvents wherein at least one stereoisomer is selectively hydrolyzed.

11. A method according to claim 10, wherein said biocatalyst is chosen from an alkaline protease, an esterase, a lipase, a hydrolase, and any combination thereof.

12. A method of preparing a stereoisomerically enriched compound of formula (II), 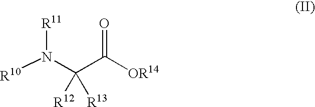
wherein:
R¹⁰ is hydrogen, —(CR¹⁵R¹⁶)ₜ(C₆-C₁₄ aryl), —CH₂CH═CH₂, —C(O)R¹⁵, —C(O)OR¹⁵, —C(O)C(O)OR¹⁵, or —Si(R¹⁵)₃, wherein said C₆-C₁₄ aryl is optionally substituted with at least one substituent chosen from halo, C₁-C₁₀ alkyl, —OR¹⁵, and —N(R¹⁵R¹⁸);
R¹¹ is hydrogen, C₁-C₁₀ alkyl, C₂-C₁₀ alkenyl, C₂-C₁₀ alkynyl, —(CR¹⁵R¹⁶)ₜ(C₆-C₁₄ aryl), or —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶);
R¹² and R¹³ are independently chosen from hydrogen, C₁-C₁₀ alkyl, C₂-C₁₀ alkenyl, C₂-C₁₀ alkynyl, —(CR¹⁵R¹⁶)ₜ(C₆-C₁₄ aryl), and —(CR¹⁵R¹⁶)(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶), provided that R¹² and R¹³ cannot both be hydrogen;
R¹⁴ is hydrogen;
R¹⁵ and R¹⁶ are independently chosen from hydrogen, halo, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5;
said method comprising:
treating a compound of formula (II), wherein R¹⁰, R¹¹, R¹², and R¹³ are as defined above, and R¹⁴ is chosen from C₁-C₁₀ alkyl, C₂-C₁₀ alkenyl, C₂-C₁₀ alkynyl, —(CR¹⁵R¹⁶)ₜ(C₆-C₁₄ aryl), and —(CR¹⁵R¹⁶)ₜ(4-10 membered heterocyclic), and wherein said C₆-C₁₄ alyl and 4-10 membered heterocyclic are optionally substituted with at least one substituent chosen from halo, C₁-C₁₀ alkyl, —OR¹⁵, and —N(R¹⁵R¹⁶), with a biocatalyst in an aqueous solution, an organic solvent, or a mixture of organic and aqueous solvents wherein at least one stereoisomer is selectively hydrolyzed.

13. A method according to claim 12, wherein in the compound of formula (Il):
R¹⁰ is —C(O)OR¹⁵ or —C(O)C(O)OR¹⁵;
R¹¹ is hydrogen or C₁-C₁₀ alkyl;
R¹² and R¹³ are independently chosen from hydrogen, C₁-C₁₀ alkyl, C₂-C₁₀ alkenyl, and C₂-C₁₀ alkynyl, provided that R¹² and R¹³ cannot both be hydrogen;
R¹⁴ is hydrogen; and
R¹⁵ is C₁-C₁₀ alkyl.

14. A method according to claim 12, wherein said biocatalyst is chosen from an alkaline protease, an esterase, a lipase, a hydrolase, and any combination thereof.

15. A method of preparing a stereoisomerically enriched compound of formula (IIA), 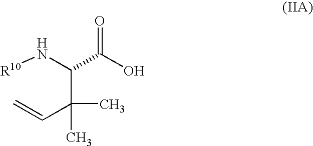
wherein:
R¹⁰ is chosen from hydrogen, —(CR¹⁵R¹⁶)ₜ(C₆-C₁₄ aryl), —CH₂CH═CH₂, —C(O)R¹⁵, —C(O)OR¹⁵, and —C(O)C(O)OR¹⁵; and
each R¹⁵ and R¹⁶ are independently chosen from hydrogen, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5;
said method comprising:
treating a compound of formula (IlB), 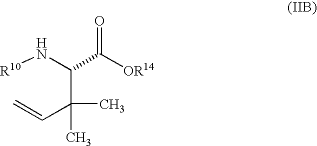
wherein R¹⁰ is as defined above, and R¹⁴ is C₁-C₁₀ alkyl, with a biocatalyst in an aqueous solution, an organic solvent, or a mixture of organic and aqueous solvents wherein at least one stereoisomer is selectively hydrolyzed.

16. A method according to claim 15, wherein said biocatalyst is chosen from an alkaline protease, an esterase, a lipase, a hydrolase, and any combination thereof.

17. A method for the resolution of a compound of formula (II), 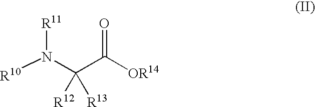
wherein:
R¹⁰ is hydrogen, —(CR¹⁵R¹⁶)ₜ(C₆-C₁₄ aryl), —CH₂CH═CH₂, —C(O)R¹⁵, —C(O)OR¹⁵—C(O)C(O)OR¹⁵, or —Si(R¹⁵)₃, wherein said C₆-C₁₄ aryl is optionally substituted with at least one substituent chosen from halo, C₁-C₁₀ alkyl, —OR¹⁵, and —N(R¹⁵R¹⁶);
R¹¹ is hydrogen, C₁-C₁₀ alkyl, C₂-C₁₀ alkenyl, C₂-C₁₀ alkynyl, —(CR¹⁵R¹⁶)ₜ(C₆-C₁₄ aryl), or —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶);
R¹² and R¹³ are independently chosen from hydrogen, C₁-C₁₀ alkyl, C₂-C₁₀ alkenyl, C₂-C₁₀ alkynyl, —(CR¹⁵R¹⁶)ₜ(C₆-C₁₄ aryl), and —(CR¹⁵R¹⁶)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁵, and —N(R¹⁵R¹⁶), provided that R¹² and R¹³ cannot both be hydrogen;
R¹⁴ is hydrogen;
R¹⁵ and R¹⁶ are independently chosen from hydrogen, halo, C₁-C₁₀ alkyl, C₁-C₁₀ alkoxy, C₂-C₁₀ alkenyl, C₂-C₁₀ alkynyl, —(CR¹⁷R¹⁷)ₜ(C₆-C₁₄ aryl), and —(CR¹⁷R¹⁷)ₜ(4-10 membered heterocyclic), wherein said C₆-C₁₄ aryl and 4-10 membered heterocyclic are optionally substituted with at least one substituent chosen from halo, C₁-C₁₀ alkyl, —OR¹⁷, and —N(R¹⁷R¹⁷);
each R¹⁷ is independently chosen from hydrogen and C₁-C₁₀ alkyl; and
t is an integer from 0 to 5;
said method comprising:
(i) treating a compound of formula (II), wherein R¹⁰, R¹¹, R¹², R¹³, and R¹⁴ are as defined above, with a chiral, non-racemic base to afford a mixture of diastereomeric salts;
(ii) separating said diastereomeric salts from each other; and
(iii) converting said diastereomeric salt to a stereoisomerically enriched compound of formula (II).

18. A method according to claim 17, wherein said chiral, non-racemic base is (R)-(−)-2-phenylglycinol or (S)-(+)-2-phenylglycinol.
